(19) United States
(12) Patent Application Publication
(10) Pub. No.: US 2009/0298169 A1
(43) Pub. Date: Dec. 3, 2009

(54) Pancreatic and Liver Endoderm Cells and Tissue by Differentiation of Definitive Endoderm Cells Obtained from Human Embryonic Stems

(75) Inventors: Stephen Dalton, Athens, GA (US); David Reynolds, Athens, GA (US); Michael Kulik, Athens, GA (US)

Correspondence Address: COLEMAN SUDOL SAPONE, P.C., 714 COLORADO AVENUE, BRIDGE PORT, CT, 06605-1601, US

(73) Assignee: The University of Georgia Research Foundation, Athens, GA (US)

(21) Appl. No.: 12/227,913

(22) PCT Filed: Jun. 4, 2007
(86) PCT No.: PCT/US2007/013137

§ 371 Date: Feb. 11, 2009

Related U.S. Application Data
(60) Provisional application No. 60/810,424, filed on Jun. 2, 2006.
(60) Provisional application No. 60/918,100, filed on Mar. 15, 2007.

(51) Int. Cl. C12N 5/08

(52) U.S. Cl. 435/366; 435/378

(57) ABSTRACT

The invention relates to methods that allow for the efficient differentiation to form pancreatic endoderm cells from pluripotent stem cells such as human embryonic stem cells and definitive endoderm cells. The invention is directly applicable to the ultimate generation of pancreatic beta cells that could be used as part of a therapy to treat or even cure diabetes. Additionally, the present invention may be used to generate liver endoderm cells from human embryonic stem cells and definite endoderm cells as well. This invention relates to a method for generating definitive endoderm and pancreatic endoderm cells from stem cells, preferably human embryonic stem cells using defined media in the absence of feeder cells. A simply two step procedure to provide pancreatic endoderm cells from embryonic stem cells represents further embodiments of the present invention.

### RELATED APPLICATIONS

This application claims the benefit of provisional application U.S. 60/810,424, entitled “Pancreatic and Liver Endoderm Cells and Tissue by Differentiation of Definitive Endoderm Cells Obtained from Human Embryonic Stems”, filed Jun. 2, 2006 and U.S. 60/918,100 entitled, “An Improved Method for the Generation of Definitive Endoderm and Pancreatic Endoderm from Human Embryonic Stem Cells, filed Mar. 15, 2007, both of which applications are incorporated by reference herein.

### FIELD OF THE INVENTION

The invention relates to methods that allow for the efficient differentiation to form pancreatic endoderm cells from pluripotent stem cells such as human embryonic stem cells and definitive endoderm cells. The invention is directly applicable to the generation of pancreatic beta cells that could be used as part of a therapy to cure diabetes. Additionally, the present invention may be used to generate liver endoderm cells from human embryonic stem cells and definite endoderm cells as well.

This invention relates to a method for generating definitive endoderm and pancreatic endoderm cells from stem cells, preferably human embryonic stem cells using defined media in the absence of feeder cells.

### BACKGROUND OF THE INVENTION

Embryonic Stem (ES) cells represent a powerful model system for the investigation of mechanisms underlying pluripotent cell biology and differentiation within the early embryo, as well as providing opportunities for genetic manipulation of mammals and resultant commercial, medical and agricultural applications. Furthermore, appropriate proliferation and differentiation of ES cells can be used to generate an unlimited source of cells suited to transplantation for treatment of diseases that result from cell damage or dysfunction. Other pluripotent cells and cell lines including early primitive ectoderm-like (EPL) cells as described in International Patent Application WO 99/53021, in vivo or in vitro derived ICM/epiblast, in vivo or in vitro derived primitive ectoderm, primordial germ cells (EG cells), teratocarcinoma cells (EC cells), and pluripotent cells derived by dedifferentiation or by nuclear transfer will share some or all of these properties and applications. Recently, a method for differentiating ES cells into definitive endoderm cells has been established. It is from human embryonic stem cells and definitive endoderm cells that the present invention has been made.

The successful isolation, long-term clonal maintenance, genetic manipulation and germ-line transmission of pluripotent cells has generally been difficult and the reasons for this are unknown. International Patent Application WO 97/32033 and U.S. Pat. No. 5,453,357 describe pluripotent cells including cells from species other than rodents. Human ES cells have been described in International Patent Application WO 00/27995, and in U.S. Pat. No. 6,200,806, and human EG cells have been described in International Patent Application WO 98/43679.

The ability to tightly control differentiation or form homogeneous populations of partially differentiated or terminally differentiated cells by differentiation in vitro of pluripotent cells has proved problematic. Current approaches can involve the formation of embryoid bodies from pluripotent cells in a manner that is not controlled and does not result in homogeneous populations. Mixed cell populations such as those in embryoid bodies of this type are generally unlikely to be suitable for therapeutic or commercial use.

The biochemical mechanisms regulating ES cell pluripotency and differentiation are poorly understood. However, limited empirical data available suggests that the continued maintenance of pluripotent ES cells under in vitro culture conditions is dependent upon the presence of cytokines and growth factors present in the extracellular serum milieu. A number of such factors such as insulin, IGF(s) and FGF(s) have been found to activate intracellular signaling events through the lipid kinase phosphatidylinositol 3-kinase (PI3-kinase) (Carpenter & Cantley, (1996) Curr. Opin. Cell. Biol., 8: 153-158). In response to the binding of these soluble factors to specific cell surface receptors, PI3-kinase is recruited to the intracellular membrane surface where it initiates a cascade of secondary signaling events leading to the functional regulation of several downstream intracellular targets that influence diverse biological processes.

Amongst the downstream targets of PI3-kinase is the protein kinase called ‘mammalian Target Of Rapamycin’ (mTOR). Stimulation of mTOR both precedes and is necessary for activation of ribosomal p70 S6 kinase, a serine/threonine kinase that is pivotal to the regulation of the protein synthetic machinery (Chung et al., (1994) Nature, 370: 71-75).

During embryonic development, the tissues of the body are formed from three major cell populations: ectoderm, mesoderm and definitive endoderm. These cell populations, also known as primary germ cell layers, are formed through a process known as gastrulation. Following gastrulation, each primary germ cell layer generates a specific set of cell populations and tissues. Mesoderm gives rise to blood cells, endothelial cells, cardiac and skeletal muscle, and adipocytes. Definitive endoderm generates liver, pancreas and lung. Ectoderm gives rise to the nervous system, skin and adrenal tissues.

There is a need, therefore, to identify methods and compositions for the production of a population of cells enriched in a cell lineage and to further differentiate definitive endoderm cells into pancreatic endoderm cells and/or liver endoderm cells and to promote the proliferation of these cells, and the products of their further differentiation.

Human pluripotent cells, including cells obtained from human umbilical blood, offer unique opportunities for investigating early stages of human development as well as for therapeutic intervention in several disease states, such as diabetes mellitus and Parkinson's disease. For example, the use of insulin-producing P-cells derived from hESCs would offer a vast improvement over current cell therapy procedures which utilize cells from donor pancreases. Currently cell therapy treatments for diabetes mellitus, which utilize cells from donor pancreases, are limited by the scarcity of high quality islet cells needed for transplant. Cell therapy for a single Type I diabetic patient requires a transplant of approximately 8×10⁸ pancreatic islet cells (Shapiro et al., 2000, N Engl J Med 343:230-238; Shapiro et al., 2001a, Best Pract Res Clin Endocrinol Metab 15:241-264; Shapiro et al., 2001b, Bmj 322:861). As such, currently, at least two healthy donor organs are required for to obtain sufficient islet cells for a successful transplant. Definitive endoderm cells which are obtained from hESCs, offer a source of starting material from which to develop substantial quantities of high quality differentiated pancreatic endoderm or liver endoderm cells for use in further differentiation and the production of differentiated cells which can be used in human cell therapies.

Human embryonic stem cells (hESCs) can be differentiated into the three germ layers (ectoderm, mesoderm and definitive endoderm) or extraembryonic endoderm depending on the culture conditions utilized (FIG. 1). hESCs have been successfully differentiated into definitive endoderm (DE) under a variety of conditions. D'Amour et al (2005) described a method using manually passaged hESCs grown on mouse embryo fibroblast (MEF) feeder layers in the presence of knockout serum replacement (KSR) medium as a starting point for differentiation. Differentiation into DE then involved the addition of Activin A (or similar factors such as Nodal) in the presence of media containing low levels of FCS or, the temporary absence of FCS. Another method developed by us, (McLean et al., 2007) utilized hESCs grown under feeder free conditions in media consisting of MEF conditioned media supplemented with Fgf2. Differentiation into DE was then promoted by addition of an inhibitor of PI3K signaling such as LY 294002 or rapamycin. Both methods generate populations of cells comprising ˜70-80% DE a judged by marker analysis for DE including CXCR4, Sox17, FoxA2 etc.

DE can be further differentiated into pancreatic endoderm (PE), a cell type that is a precursor for pancreatic cell lineages and expresses markers such Pdx1. In transition from DE to PE, cells pass through a gut tube like state. Methods for PE formation have been described from hESCs grown on MEF feeder layers that were passaged manually. Differentiation from DE to PE involved cells transitioning through a gut tube state where cells expressed markers such as Tcf2/HNF1B and HNF4A and involves culture in FCS for at least part of the differentiation (D'Amour et al., 2006). We have also described methods for PE formation by addition of retinoic acid directly from DE cultures that also involves culture in FCS on Matrigel for collagenase passaged cells (Dalton and Kulik UGARF filing 2006).

D'Amour et al. Nature Biotech, 2005

### McLean et al., Stem Cells, January 2007

### D'Amour et al., Nature Biotech, 2006

As described above, several methods have been reported in patent filings or peer reviewed publications for the generation of DE from hESCs. These methods use either feeder fibroblasts or feeder free conditions but always in the presence of fetal calf serum and/or KSR. This is problematic because these components generate experimental inconsistencies due to batch variations. Since FCS and KSR contain undefined activities this is problematic when using hESCs for therapeutic development.

### BRIEF DESCRIPTION OF THE FIGURES

FIG. 1 shows the generation of cells expressing the embryonic liver marker alphafetoprotein (AFP) following treatment of definitive endoderm. BG01 hESCs were differentiated into definitive endoderm for four days following the addition of LY 294002 (50 μM). Media was changed to DMEM/F12, 10% FCS and cells grown for up to six more days. Untreated: untreated hESCs. AFP transcript levels were analyzed by QRT-PCR in triplicate and shown as the fold-increase over untreated samples (hESCs) after normalization to GAPDH reference transcript. Note: this result can be achieved in the presence of absence of Fgf10 although optimal AFP induction is seen following addition of Fgf10.

FIG. 2 shows the time course of Pdx1 transcript induction following RA treatment. BG01 hESCs were treated with LY 294002 (50 μM) for four days then switched to media containing DMEM/F12, 10% FCS, 50 ng/ml Fgf10 and 2 μM retinoic acid for up to four days. Untreated-untreated hESCs. Transcript levels were analyzed by QRT-PCR in triplicate and shown as the fold-increase over untreated samples (hESCs) after normalization to GAPDH reference transcript. Fold-induction of Pdx1 transcript levels are indicated.

FIG. 3 shows the time course of Pdx1 and Isl1 transcript induction following RA treatment. BG01 hESCs were treated with LY 294002 (50 μM) for four days then switched to media consisting of DMEM/F12, 10% FCS, 50 ng/ml Fgf10 and 2 μM retinoic acid for up to four days. Untreated: untreated hESCs. Transcript levels were analyzed by QRT-PCR in triplicate and shown as the fold-increase over untreated samples (hESCs) after normalization to GAPDH reference transcript.

FIG. 4 shows the changes in Sox17, AFP and Pdx1 in response to different culture conditions. Untreated: untreated BG01 hESCs cultured on MatriGel in the presence of MEF-CM, Fgf2, 20% KSR. LYA: hESCs grown on Matrigel in MEF-CM and Fgf2 were treated with LY 294002 for four days. F106d: hESCs treated with LY 294002 for four days were switched to media containing Fgf10 (50 ng/ml), 10% FCS for a further six days. RA4d/2d: hESCs treated with LY 294002 for 4 days were switched to media (DMEM/F12) containing Fgf10 (50 ng/ml), 2 μM RA, 10% FCS for a further four days. This was followed by culturing for a further two days in the same media lacking RA and Fgf10, Sox17, AFP and Pdx1 transcripts were analyzed by QRT-PCR in triplicate and shown as the fold-increase over untreated samples (hESCs) after normalization to GAPDH reference transcript.

FIG. 5 shows the immunofluorescence staining of Pdx1+ cells treated with RA. BG01 hESCs were differentiated into definitive endoderm for four days following the addition of LY 294002 (50 μM). Media was changed to DMEM/F12, 10% FCS and cells grown for up to five more days as follows. hESCs treated with LY 294002 for 4 days were switched to media (DMEM/F12) containing Fgf10 (50 ng/ml), 2 μm RA, 10% FCS for a further five days. Treated and untreated (hESCs) were grown in LabTec chamber slides, fixed with 4% paraformaldehyde and probed a rabbit anti-human Pdx1 antibody (Chemicon, 1:1,000) followed by AlexaFluor (594 nm) labeled goat anti-rabbit secondary antibody (red). Cells were mounted in media containing DAPI for visualization of nuclear DNA (blue).

FIG. 6 shows that hESCs expressing markers such as Oct4, Nanog, Sox2 and Rex1 can be differentiated into the three germ layers (mesoderm, ectoderm and definitive endoderm) or extraembryonic cell types, when cultured under the appropriate conditions. To generate definitive endoderm, hESCs transition first through a mesendoderm-like state (T+, MixL1+, Wnt3a+). After transitioning through mesendoderm cells can become definitive endoderm (CXCR4+, Sox17+, GATA4,6+, Gsc+, FoxA2+). Definitive endoderm is the precursor cell type that can give rise to other endoderm lineages.

FIG. 7 shows BG02 hESCs which were passaged with accutase in hESC defined media formulation (a). For DE differentiation, media was replaced after ˜18-24 hours with differentiation media (a), in the presence or absence of Wnt3a (25 ng/ml). RNA was prepared from cultures at times indicated and subject to QRT-PCR analysis of T, MixL1, GSC, Sox17 and CXCR4 transcripts. QRT-PCR reactions are normalized to GAPDH control. ‘Assays on demand’ QRT-PCR reactions were from Applied Biosystems and have been described previously (McLean et al., 2007).

FIG. 8 shows that definitive endoderm differentiation occurs at the exclusion of other cell lineages. BG02 hESCs were plated in defined conditions (a) and after 18 hours media was switched to differentiation media (a) with Wnt3a (25 ng/ml) for the first 24 hours. The time course proceeded for 96 hours. Untreated hESCs (96 hours) or cells in differentiation media (a) were collected at 24, 48, 72 and 96 hours for QRT-PCR analysis. ‘Assays on demand’ QRT-PCR reactions were from Applied Biosystems and have been described previously (McLean et al., 2007).

FIG. 9 shows BG02 hESCs which were plated and differentiated as described in FIG. 8. At times indicated (UT, untreated; 24, 48, 72, 96 hours in differentiation medium [a]), cells were fixed and subject to immunocytochemistry (ICC) by probing with antibodies that recognize T (R&D Systems) and Sox17 (D'Amour et al., 2005). DNA is stained with DAPI and a merge of staining for DAPI, T and Sox17 is shown. Magnification 20×.

FIG. 10 shows that nanog positive cells decrease during differentiation. BG02 hESCs were plated and differentiated as described for FIGS. 8, 9. At times indicated (UT, untreated; 24, 48, 72, 96 hours in differentiation medium [a]), cells were fixed and subject to immunocytochemistry (ICC) by probing with antibodies that recognize Nanog. DNA is stained with DAPI and a merge of staining for DAPI and Nanog is shown. Magnification 20×.

FIG. 11 shows BG02 hESCs which were differentiated for 96 hours in differentiation medium (a) and stained with an antibody that recognizes the DE cell surface marker CXCR4. Untreated HESC cultures (BG02) had only a very small % of CXCR4+ cells (<3%) but >93% of treated cells (BG02 d4-DE) were positive for CXCR4.

FIG. 12 shows bright field pictures of BG02 hESCs, DE produced in defined conditions. DE was produced over a 4 day period. 10× magnification.

FIG. 13 shows a QRT-PCR analysis of transcripts associated with PE formation in defined media conditions after treatment of DE with RA. BG02 hESCs were differentiated into DE over 4 days, split and differentiated in media containing retinoic acid and Fgf10 for the times indicated. QRT-PCR data is shown after normalization to GAPDH control. Time points indicate time after DE formation (days).

FIG. 14 shows ICC of PE cultures produced from hESC-derived DE under defined conditions. Cells were differentiated as described in the legend to FIG. 13. Cells were fixed with 4% paraformaldehyde then permeabilized with Triton X-100 at days 2, 6, 12 after treatment with RA and Fgf10 then stained with a goat anti-human Pdx1 antibody or TCF2 antibodies (R&D Systems) and DAPI (DNA). Magnification 20×.

FIG. 15 shows the differentiation of hESC's to pancreatic endoderm cells. These cells are differentiated in defined conditions as indicated for 3-5 days for step 1 (Activin A, low P13Kinase or P13Kinase inhibitor) followed by 8-10 days (step 2) the time indicated in the figure and the levels of Ngn3 and Pdx1 mRNAs evaluated using Q-PCR, using TaqMan probes.

### BRIEF DESCRIPTION OF THE INVENTION

The present invention relates to pancreatic endoderm cells (PE) which are obtained from definitive endoderm cells by exposing the definitive endoderm cells to an effective concentration of retinoic acid (at least about 0.05-0.1 μg/ml, preferably about 0.1-25 μg/ml, more preferably about 0.1-2.0 μg/ml) in base media comprising an effective amount of fetal calf serum (FCS— preferably about 10% of the base media plus serum) for a period of at least about 2 days, preferably at least about 4 days, and more preferably about 4 days, followed by exposing the cells obtained from the first step to FCS (preferably, about 10%) in base media for at least one day, preferably at least about 2 days in the absence of retinoic acid. This method preferably results in at least about 35-50% and preferably at least about 70-80+% of the cells in the treated sample expressing the pancreatic endoderm markers, Pdx1 and Isl1.

Using the above-described method the present invention generates a population of cells from definitive endoderm cells that are up to 70-80% pure for the PE markers, Pdx1 and Isl1. Following treatment of human definitive endoderm cells (DE), routinely observed increases in Pdx1 mRNA ranging from 70-fold to several hundred-fold using the method of the present invention occur. This efficiency of production represents an unexpected result. In prior art methods, the efficiency of production of PE (Pdx1+ and lsl1+) from DE ranges is generally about 10-20%.

In an alternative embodiment, the definitive endoderm (DE) cells may be differentiated into liver endoderm cells by following the same method which is utilized above for the preparation of pancreatic endoderm cells, but in this method aspect substituting Fgf10 in an effective amount 10 ng/ml-100 ng/ml (preferably, about 50 ng/ml) for the retinoic acid.

Although any definitive endoderm (DE) may be used in the present invention, the preferred endoderm is that obtained from human embryonic stem cells using the PI3K inhibitor LY 294002. In a preferred method for obtaining DE from hESC, hESC's are exposed to LY 294002 for a period of about 4-5 days in a basal media to obtain the definitive endoderm which is preferably used to produce pancreatic endoderm expressing the biomarker

DE is a human cell type with the capacity to differentiate into cells including those of the liver, lung, pancreas, thymus, intestine, stomach and thyroid. In the present invention we have been able to generate pancreatic endoderm (PE) cells in our approach to ultimately provide pancreatic beta cells in a highly efficient, consistent manner. The present method establishes a convenient approach for the differentiation of definitive endoderm into pancreatic endoderm (PE). Pancreatic endoderm is a cell type capable of differentiating into multiple pancreatic lineages, including beta cells, but no longer has the capacity to differentiate into non-pancreatic lineages.

In one aspect, the present invention relates to a population of cells which are up to 70-80+% type for the pancreatic endoderm marker, Pdx1 and/or Isl1. In the present invention, following treatment of human embryonic stem cells (hESCs) to form definitive endoderm (DE) cells, the definitive endoderm cells are exposed to an effective concentration of retinoic acid in base media optionally comprising fetal calf serum (FCS) for a period of at least about 2 days, preferably at least 4 days, and more preferably about 4 days, followed by contacting the differentiated cells from the first step with base media (preferably comprising an effective amount of FCS) for at least one day, preferably at least about 2 days in the absence of retinoic acid. This method preferably results in at least about 50% and preferably at least about 70-80+% of the cells in the treated sample expressing the pancreatic endoderm market, Pdx1 and/or Isl1.

In addition to the above-described preferred method for producing the starting definitive endoderm cells, in the present invention, definitive endoderm cells may be produced by any method known in the art, including, for example the methods which are set forth in United States application publication 20060003446 to G. Keller, et al.; 20060003313 to K. D'Amour, et al., 20050158853 to K. D'Amour, et al., and 20050260749 of Jon Odorico, et al., relevant portions of which are incorporated by reference herein.

In alternative embodiments, the present invention relates to methods and conditions for the improved differentiation of hESCs into DE and then PE using defined media that does not use an undefined component such as fetal calf serum or serum supplements such as KSR medium. The efficiency of DE production and the robustness of the culture system is significantly greater than that reported previously.

In these alternative embodiments, the present invention relates to a method of generating definitive endoderm cells from mammalian embryonic stem cells, preferably human embryonic stem cells, under feeder cell-free conditions, comprising exposing plated embryonic stem cells to a defined media or MEF conditioned media using a growth matrix or as otherwise described herein, and thereafter, the stem cells are exposed to a differentiation media which is a defined media (with an absence of fetal calf serum or KSR-type serum components and an absence of IGF and insulin) comprising an effective amounts of an activator of the SMAD pathway, such as a TGFβ super family member (concentration of 1 ng/ml to 100 ng/ml, preferably about 25-50 ng/ml), e.g., Activin A, nodal, TGFβ or other TGF component and optionally, an inhibitor of PI3kinase signaling (as otherwise described herein). Optionally, an effective amount of bovine serum albumin is included (about 0.5-3%, preferably about 2%) to provide a protein source. Preferably, the defined media contains an inhibitor of P13K signaling. Preferably, an effective amount of Wnt3a is included (1 ng/ml to about 100 ng/ml, preferably about 25 ng/ml. Preferably, the growth matrix, as otherwise described herein, is matrigel.

In this aspect, the method of the present invention provides high levels of definitive endoderm from human embryonic stem cells (grown on essentially any medium, preferably a defined medium) by exposing the stem cells to a defined medium which includes a component which promotes differentiation to definitive endoderm cells (using effective amounts of Activin A, nodal or TGFβ or as otherwise described above) in the absence of serum or factors (IGF or insulin) or components which promote PI3Kinase activity. The definitive endoderm cells obtained after about 3-6 days, preferably 4-5 days, may thereafter be exposed to effective concentrations of retinoic acid (at least about 0.1-0.2 μg/ml, preferably at least about 1 μg/ml, about 2-25 μg/ml, more preferably about 10 μg/ml and optionally, an effective amount of Fgf10 (at a concentration of about 1 ng/ml to about 100 ng/ml, with a preferred concentration of about 50 ng/ml) in a defined medium (preferably containing Wnt3a and BSA) for a period of about 5-12 days, preferably about 8-10 days, to produce pancreatic endoderm (PE) cells exhibiting Pdx1 and Isl1 markers which may be further exposed to the same medium for an additional several days (about 1-5 days) to produce Endocrine pancreas cells which exhibit Ngn3 and Nkx6.1 markers.

This invention is applicable to the culture of hESCs grown under a wide variety of feeder free conditions including, but not restricted to, cells grown in;

1. MEF conditioned media on matrices such as Matrigel, which contain a differentiation agent;
2. defined media formulation that does not use conditioned media, FCS or KSR-type serum replacements

As otherwise described herein, also useful in place of Matrigel are BD Cell-Tak™ Cell and Tissue Adhesive, BD™ FIBROGEN Human Recombinant Collagen I, BD™ FIBROGEN Human Recombinant Collagen m, BD Matrigel™ Basement Membrane Matrix, BD Matrigel™ Basement Membrane Matrix High Concentration (HC), BD™ PuraMatrix™ Peptide Hydrogel, Collagen I, Collagen I High Concentration (HC), Collagen II (Bovine), Collagen III, Collagen IV, Collagen V, and Collagen VI, among others, which contain effective amounts of one or more of laminin, tenascin, thrombospondin, collagen, fibronectin, vibronectin, polylysine, polyornithine and mixtures thereof.

The invention also relates to a method of generating definitive endoderm cells from embryonic stem cells, preferably human embryonic stem cells, comprising exposing the embryonic stem cells in the absence of feeder cells to a differentiation media comprising elevated levels of Activin A, nodal or TNFβ and optionally, an inhibitor of PI3kinase signaling, wherein the differentiation media is a defined media free from fetal calf serum or KSR-type serum components.

In this aspect of the invention, the definitive endoderm cells are produced at a level of at least about 90% from the embryonic stem cells.

Definitive endoderm cells in each of the embodiments of the present invention or otherwise available from the art may be further differentiated into pancreatic endoderm cells.

### DETAILED DESCRIPTION OF THE INVENTION

The following terms shall be used to describe the present invention:

Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5th Ed., Berlin: Springer-Verlag; and in Current Protocols in Molecular Biology, F. M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “a cell” can mean that at least one cell can be utilized.

The present invention may be understood more readily by reference to the following detailed description of the preferred embodiments of the invention and the Examples included herein. However, before the present compositions and methods are disclosed and described, it is to be understood that this invention is not limited to specific specific conditions, or specific methods, etc., as such may, of course, vary, and the numerous modifications and variations therein will be apparent to those skilled in the art.

Standard techniques for growing cells, separating cells, and where relevant, cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth. Enzymol. 68; Wu et al., (Eds.) 1983 Meth. Enzymol. 100 and 101; Grossman and Moldave (Eds.) 1980 Meth. Enzymol. 65; Miller (ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology, Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

As used herein, the terms “differentiation agent” refers to any compound or molecule that induces a cell such as hESC's or definitive endoderm cells to partially or terminally differentiate, wherein said differentiation is due at least in part to inhibition of signaling through the PI3-kinase pathway (for the formation of definitive endoderm cells), the inclusion of effective amounts of retinoic acid to form pancreatic endoderm cells or the inclusion of effective amounts of a fibroblast growth factor, such as fibroblast growth factor 10 (Fgf10) for the formation of liver endoderm cells. While the differentiation agent may be as described below, the term is not limited thereto. The term “differentiation agent” as used herein includes within its scope a natural or synthetic molecule or molecules which exhibit(s) similar biological activity.

As used herein, the term “inhibitor of the PI3-kinase pathway” refers to any molecule or compound that decreases the activity of PI3-kinase or at least one molecule downstream of PI3-kinase in a cell contacted with the inhibitor. These inhibitors are preferred inhibitors for preparing definitive endoderm cells which are starting material cells for use in the present invention. The term encompasses, e.g., PI3-kinase antagonists, antagonists of the PI3-kinase signal transduction cascade, compounds that decrease the synthesis or expression of endogenous PI3-kinase, compounds that decrease release of endogenous PI3-kinase, and compounds that inhibit activators of PI3-kinase activity. In certain embodiments of the foregoing, the inhibitor is selected from the group consisting of Rapamycin, LY 294002, wortmanin, lithium chloride, Akt inhibitor I, Akt inhibitor II (SH-5), Akt inhibitor III (SH-6), NL-71-101, and mixtures of the foregoing. Akt inhibitor I, II, Akt III, and NL-71-101 are commercially available from Calbiochem. In other embodiments, the inhibitor is selected from the group consisting of Rapamycin and LY 294002. In a further preferred embodiment, the inhibitor comprises LY 294002. In another embodiment, the inhibitor comprises Akt1-II. It is understood that combinations of inhibitors may be used to elicit the desired differentiation effect. The ultimate result is production of substantial quantities of definitive endoderm cells for use as a starting cell line for the production of pancreatic endoderm cells and/or liver endoderm cells according to the present invention.

In one preferred embodiment, the pluripotent hESC cells are contacted with an effective amount of an inhibitor of the PI3-kinase pathway (preferably LY 294002, also known as [2-(4-Morpholinyl)-8-phenyl-4H-1-benzopyran-4-one, available from Calbiochem, among numerous other biochemical manufacturers) to produce definitive endoderm cells, and the definitive endoderm cells so produced are contacted with an effective amount of retinoic acid (about 0.005-25 μg/ml, more preferably about 0.1-2.0 μg/ml, more preferably about 0.2-2.0 μg/ml) in base media comprising fetal calf serum (about 1.0% to about 20%, preferably about 10% fetal calf serum). The definitive endoderm cells are exposed to the retinoic acid containing media for a period of at least about 2 days, preferably at least 4 about days, and more preferably about 4 days, after which time the cells are exposed to media comprising FCS (preferably, about 10%) in base media for at least one day, preferably at least about 2 days in the absence of retinoic acid. The cells are preferably separated at each step, but may be simply carried forward without separation. This method preferably results in at least about 30%, at least 40%, at least 50%, at least 60% and preferably at least about 70-80+% of the cells in the treated sample expressing the pancreatic endoderm market, Pdx1 and/or Isl1.

Using the above-described method the present invention generates a population of cells from definitive endoderm cells that are up to 70-80% pure for the PE marker, Pdx1 and Isl1. Following treatment of human embryonic stem cells (hESCs), routinely observed are increases in Pdx1 mRNA expression ranging from 70-fold to several hundred-fold using the method of the present invention. Also there is a marked increase in Isl1. This efficiency of production represents an unexpected result. In prior art methods, the efficiency of production of PE (Pdx1+ and Isl1+) from hESC's ranges from about 10-20%.

In an alternative embodiment, the definitive endoderm (DE) cells may be differentiated into liver endoderm (LE) cells by following the same method which is utilized above for the preparation of pancreatic endoderm cells, but instead substituting a fibroblast growth factor, preferably Fgf10 in an effective amount, preferably about 10 ng/ml-100 ng/ml, preferably, about 25 ng/ml-75 ng/ml, more preferably, about 50 ng/ml, for the retinoic acid. In this method, definitive endoderm cells are exposed to any one or more of the fibroblast growth factors, preferably Fgf10, in a basal media preferably comprising FCS (about 1% to about 20%, preferably about 10% FCS in the basal media), for a period of at least a day, preferably at least about 2 days, even more preferably at least about 4 days or more, or preferably about 4 days, followed by exposing the cells from the first step to basal cell media optionally comprising fetal calf serum in an effective amount (about 1% to about 20%, preferably about 10% FCS) and an absence of fibroblast growth factor, for a period of at least one day and preferably at least about 2 days, or preferably about 2 days.

In alternative embodiments, the present invention relates to methods and conditions for the improved differentiation of hESCs into DE and then PE using defined media that does not use an undefined component such as fetal calf serum or serum supplements such as KSR medium. The efficiency of DE production and the robustness of the culture system is significantly greater than that reported previously.

In these alternative embodiments, the present invention relates to a method of generating definitive endoderm cells from mammalian embryonic stem cells, preferably human embryonic stem cells, under feeder cell-free conditions, comprising exposing plated embryonic stem cells to a defined media or MEF conditioned media using a growth matrix or as otherwise described herein, and thereafter, the stem cells are exposed to a differentiation media which is a defined media (with an absence of fetal calf serum or KSR-type serum components and an absence of IGF and insulin) comprising an effective amounts of an activator of the SMAD pathway, such as a TGFβ super family member (concentration of 1 ng/ml to 100 ng/ml, preferably about 25-50 ng/ml), e.g., Activin A, nodal, TGFβ or other TGF component and optionally, an inhibitor of PI3kinase signaling (as otherwise described herein). Optionally, an effective amount of bovine serum albumin is included (about 0.5-3%, preferably about 2%) to provide a protein source. Preferably, the defined media contains an inhibitor of P13K signaling. Preferably, an effective amount of Wnt3a is included (1 ng/ml to about 100 ng/ml, preferably about 25 ng/ml. Preferably, the growth matrix, as otherwise described herein, is matrigel.

In this aspect, the method of the present invention provides high levels of definitive endoderm from human embryonic stem cells (grown on essentially any medium, preferably a defined medium) by exposing the stem cells to a defined medium which includes a component which promotes differentiation to definitive endoderm cells (using effective amounts of Activin A, nodal or TGFβ or as otherwise described above) in the absence of serum or factors (IGF or insulin) or components which promote PI3Kinase activity. The definitive endoderm cells obtained after about 3-6 days, preferably 4-5 days, may thereafter be exposed to effective concentrations of retinoic acid (at least about 0.1-0.2 μg/ml, preferably at least about 1 μg/ml, about 2-25 μg/ml, more preferably about 10 μg/ml and optionally, an effective amount of Fgf10 (at a concentration of about 1 ng/ml to about 100 ng/ml, with a preferred concentration of about 50 ng/ml) in a defined medium (preferably containing Wnt3a and BSA) for a period of 8-10 days to produce pancreatic endoderm (PE) cells exhibiting Pdx1 and Isl1 markers which may be further exposed to the same medium for an additional several days (about 1-5 days) to produce endocrine pancreas cells which exhibit Ngn3 and Nkx6.1 markers.

In each of the above methods, the cells may be separated by passaging with trypsin or accutase or similar reagent, isolated and carried forward in the method aspect of the present invention or alternatively, and preferably, the cells from each step which are produced in layers are carried forth to the next step without further separation. The cells may also be centrifuged and pelleted prior to use in order to limit the size of the cell samples to single cells or clusters containing fewer cells.

As used herein, the term “effective amount” refers to that amount or concentration of any component or material which is used to produce an intended result in the present invention. The term may apply to a P13-kinase inhibitor such as LY294002 which may be used advantageously to produce definitive endoderm cells, to retinoic acid which is used as a differentiation agent to produce pancreatic endoderm cells from definitive endoderm cells or fibroblast growth factor which is used as a differentiation agent to produce liver endoderm cells from definitive endoderm cells, etc.

The term “retinoic acid’ refers to all-trans retinoic acid.

The term fibroblast growth factor of Fgf refers to a growth factor which is used to in the absence of retinoic acid in the method of the present invention to produce liver endoderm cells. Although any one or more of the various fibroblast growth factors may be used in the method of the present invention, the preferred fibroblast growth factor is fibroblast growth factor 10 (Fgf 10).

The term “basal cell medium” “basal cell media” or “basal media” or “cell differentiation medium” or “stabilizing medium” is used to describe a cellular growth medium in which the definitive endoderm cells are produced or alternatively, are differentiated into pancreatic endoderm (PE) cells or liver endoderm (PE) cells or are stabilized after they are differentiated. Basal cell media are well known in the art and comprise at least a minimum essential medium plus optional components such as growth factors, including fibroblast growth factor, retinoic acid, glucose, non-essential amino acids, salts (including trace elements), glutamine, insulin (where indicated and not excluded), transferrin, beta mercaptoethanol, and other agents well known in the art and as otherwise described herein. Preferred media includes basal cell media which contains between 2% and 20% (preferably, about 10%) fetal calf serum, or for defined medium an absence of fetal calf serum and KSR, but including bovine serum albumin). DMEM/F12 is a particularly preferred basal cell media which contains 10% FCS. Basal cell media useful in the present invention are commercially available and can be supplemented with commercially available components, available from Invitrogen Corp. (GIBCO), Cell Applications, Inc. and Biological Industries, Beth HaEmek, Israel, among numerous other commercial sources. In preferred embodiments at least one differentiation agent such as retinoic acid, fibroblast growth factor or LY294002 is added to the cell media in which a stem cell or progenitor cell is grown in order to promote differentiation of the stem cells into progenitor cells and the progenitor cells into pancreatic endoderm cells or liver cells or stem cells into pancreatic endoderm cells or liver cells. One of ordinary skill in the art will be able to readily modify the cell media to produce progenitor or pancreatic/liver cells pursuant to the present invention. Cell differentiation medium is essentially synonymous with basal cell medium but is used within the context of a differentiation process and includes cell differentiation agents to differentiate cells into other cells. Stabilizing medium is a basal cell medium which is used either before or after a differentiation step in order to stabilize a cell line for further use. In general, as used herein, cell differentiation medium and stabilizing medium may include essentially similar components of a basal cell medium, but are used within different contexts and may include different components in order to effect the intended result of the use of the medium.

In a preferred the case of forming pancreatic endoderm cells in defined medium (“defined medium”), the medium is a defined minimum essential medium (DMEM/F12 50:50 from Gibco is preferred) which excludes fetal calf serum or KSR (knockout serum replacement), insulin and IGF, but includes a SMAD pathway activator, such as a TGFβ super family member (concentration of 1 ng/ml to 100 ng/ml, preferably about 25-50 ng/ml), e.g., Activin A, nodal, TGFβ or other TGF component and optionally, an effective amount of an inhibitor of PI3kinase signaling (as otherwise described herein). Optionally, an effective amount of bovine serum albumin is included (about 0.5-3%, preferably about 2%) to provide a protein source. Preferably, the defined media contains an inhibitor of P13K signaling. Preferably, an effective amount of Wnt3a is included (1 ng/ml to about 100 ng/ml, preferably about 25 ng/ml. Preferably, the growth matrix, as otherwise described herein, is matrigel.

The cells are preferably grown on a cellular support. In the present invention, the use of Matrigel as a cellular support is preferred. Cellular supports preferably comprise at least one differentiation protein. The term “differentiation protein” is used to describe a protein which is used to grow cells to promote differentiation (also preferably attachment) of a embryonic stem cell or definitive endoderm cell. Differentiation proteins which are preferably used in the present invention include, for example, an extracellular matrix protein, which is a protein found in the extracellular matrix, such as laminin, tenascin, thrombospondin, and mixtures thereof, which exhibit growth promoting and contain domains with homology to epidermal growth factor (EGF) and exhibit growth promoting and differentiation activity. Other differentiation proteins which may be used in the present invention include for example, collagen, fibronectin, vibronectin, polylysine, polyornithine and mixtures thereof. In addition, gels and other materials which contain effective concentrations of one or more of these embryonic stem cell differentiation proteins may also be used. Exemplary embryonic stem cell differentiation proteins or materials which include these differentiation proteins include, for example, BD Cell-Tak™ Cell and Tissue Adhesive, BD™ FIBROGEN Human Recombinant Collagen I, BD™ FIBROGEN Human Recombinant Collagen III, BD Matrigel™ Basement Membrane Matrix, BD Matrigel™ Basement Membrane Matrix High Concentration (HC), BD™ PuraMatrix™ Peptide Hydrogel, Collagen I, Collagen I High Concentration (HC), Collagen II (Bovine), Collagen III, Collagen IV, Collagen V, and Collagen VI, among others. The preferred differentiation protein material for use in the present invention includes the Matrigel™ materials.

A preferred composition/material which contains one or more differentiation protein is BD Matrigel™ Basement Membrane Matrix. This is a solubilized basement membrane preparation extracted from the Engelbreth-Holm-Swarm (EHS) mouse sarcoma, a tumor rich in ECM proteins. Its major component is laminin, followed by collagen IV, heparan sulfate, proteoglycans, entactin and nidogen.

As used herein, the term “activate” refers to an increase in expression of Pdx1 or Isl1 or an upregulation of the activity of Pdx1, Isl1 or a liver marker.

As used herein when referring to a cell, cell line, cell culture or population of cells, the term “isolated” refers to being substantially separated from the natural source of the cells such that the cell, cell line, cell culture, or population of cells are capable of being cultured in vitro. In addition, the term “isolating” is used to refer to the physical selection of one or more cells out of a group of two or more cells, wherein the cells are selected based on cell morphology and/or the expression of various markers.

As used herein, the term “express” refers to the transcription of a polynucleotide or translation of a polypeptide in a cell, such that levels of the molecule are measurably higher in a cell that expresses the molecule than they are in a cell that does not express the molecule. Methods to measure the expression of a molecule are well known to those of ordinary skill in the art, and include without limitation, Northern blotting, RT-PCT, in situ hybridization, Western blotting, and immunostaining.

As used herein, the term “contacting” (i.e., contacting a definitive endoderm cell, with a compound) is intended to include incubating the compound and the cell together in vitro (e.g., adding the compound to cells in culture). The term “contacting” is not intended to include the in vivo exposure of cells to a retinoic acid, fibroblast growth factor or other differentiation agent such as an inhibitor of the PI3-kinase pathway that may occur naturally in a subject (i.e., exposure that may occur as a result of a natural physiological process). The step of contacting the cell with retinoic acid or fibroblast growth factor, or in the case of the production of definitive endoderm cells, an inhibitor of the PI3-kinase pathway such as LY294002 can be conducted in any suitable manner. For example, the cells may be treated in adherent culture, or in suspension culture. It is understood that the cells contacted with the differentiation agent may be further treated with other cell differentiation environments to stabilize the cells, or to differentiate the cells further, for example to produce islet cells.

Applicant has demonstrated that culturing definitive endoderm cells with retinoic acid in basal cell media generates differentiated cells as pancreatic or liver endoderm cells wherein the cells have greater homogeneity than spontaneously differentiated cells.

The present invention contemplates a composition comprising a population of isolated differentiated mammalian cells, preferably human pancreatic endoderm cells, wherein the cells are differentiated from a pluripotent or definitive endoderm cell in vitro, and wherein greater than approximately 30% of the cells express Pdx1 and/or Isl1. In one embodiment of the inventions greater than approximately 35%, 40%, 45%, 50%, 55%, 60%, 65%, 67%, 70%, 72%, 74%, 75%, 76%, 77%, 78%, 79% or even 80% of the cells express Pdx1 and/or Isl1. Preferably, at the composition comprises a population of cells at least 50% of which express Pdx1 and/or Isl1, up to 70-80% or more.

The invention further contemplates a composition comprising a homogenous population of isolated liver endoderm cells, wherein the cells were differentiated in an in vitro culture, and wherein greater than approximately 35%, 40%, 45%, 50%, 55%, 600%, 65%, 67%, 70%, 72%, 74%, 75%, 76%, 77%, 78%, 79% or even 80% of the cells are liver endoderm cells.

The invention further encompasses a method of differentiating a pluripotent mammalian cell, preferably a human pluripotent cell, into a pancreatic endoderm cell comprising: (a) providing the pluripotent mammalian cell, and (b) contacting the pluripotent mammalian cell with an effective amount of an inhibitor of the PI3-kinase signaling pathway to at least partially differentiate the pluripotent cell to a cell of a definitive endoderm lineage and thereafter, differentiating the definitive endoderm cells to pancreatic endoderm cells using retinoic acid as the differentiation agent in a basal medium (preferably DMEM/F12) comprising an effective amount of FCS (preferably about 10%). The endoderm cells may be isolated at that time, but preferably are exposed to DMEM/F12, optionally included FCS (preferably, about 10%) in the absence of retinoic acid for a further day or more (preferably, two days) wherein the pancreatic endoderm cells are isolated. The pancreatic endoderm cells may be further differentiated into pancreatic β cells.

In an alternative embodiment of the present invention, the production of liver endoderm cells occurs following the same steps above, but instead of differentiating the definitive endoderm cells with retinoic acid, retinoic acid is avoided and differentiation occurs in the presence of fibroblast growth factor (Fgf 10) whereupon liver endoderm cells are produced instead of pancreatic endoderm cells.

As described, the pancreatic endoderm cells may be further differentiated into pancreatic β-cells and used in the treatment of diabetes mellitus (type I).

It is contemplated that the definitive endoderm cells are differentiated by contact with retinoic acid to produce pancreatic endoderm cells. In one embodiment, the cells are dissociated to an essentially single cell culture prior to being contacted with the retinoic acid in basal cell media. The cells can be dissociated using a protease, such as, but not limited to, trypsin. In one embodiment, the cells are contacted with the retinoic acid after being plated for between approximately 12 hours to approximately 6 days, after being plated for between approximately 12 hours to approximately 48 hours, or after being plated for approximately 24 hours. In one embodiment, the cells are contacted with the retinoic acid for greater than approximately 24 hours, for greater than approximately 48 hours, for greater than approximately 72 hours, for greater than approximately 96 hours, or for approximately 96 hours. After exposure to retinoic acid in basal cell media, the pancreatic endoderm cells obtained may be separated directly (trypsinized) and then optionally and preferably exposed to basal cell media (optionally comprising FCS) in the absence of retinoic acid for at least 12 hours, for at least 24 hours, for at least 48 hours or 48 hours, or alternatively the pancreatic endoderm cells obtained from exposure to retinoic acid may be further exposed to basal cell media in the absence of retinoic acid without separation.

It is preferred that the retinoic acid is effective in causing differentiation of the definitive endoderm cell towards a pancreatic endodermal lineage after the cell has been cultured with the composition for greater than approximately 24 hours, preferably at least 48 hours. It is also contemplated that the retinoic acid is effective in causing differentiation of a pluripotent mammalian cell towards an endodermal lineage when the cell has been plated for greater than approximately 12 hours before it is contacted with the inhibitor, or when the cell has been plated for approximately 24 hours before it is contacted with the inhibitor.

In certain embodiments, the definitive endoderm cells are plated at a concentration of less than approximately 2.5×10⁶ cells/35 mm dish, of at least approximately 2.5×10⁴ cells/35 mm dish, between approximately 2.5×10⁵ to approximately 2×10⁶ cells/35 mm dish, between approximately 5×10⁵ to approximately 2×10⁶ cells/35 mm dish, of less than approximately 2×10⁶ cells/35 mm dish, or at a density of greater than 4×10⁵ cells/35 mm dish. In certain preferred aspects, the definitive cells are plated at a concentration of approximately 7.5×10⁵ cells/35 mm dish.

In producing definitive endoderm cells from pluripotent cells, in particular human embryonic stem cells (hESC), as a first step in alternative embodiments of the present invention, the present invention further encompasses the use of a composition for culturing cells, comprising a cell culture medium and a differentiation agent which is an inhibitor of the PI3-kinase pathway to differentiate embryonic stem cells into definitive endoderm cells. In certain embodiments of the invention, the inhibitor is selected from the group consisting of LY 294002, Rapamycin, wortmanin, lithium chloride, Akt inhibitor I, Akt inhibitor II, Akt inhibitor III, NL-71-101, and mixtures of the foregoing. In one embodiment, the inhibitor is Rapamycin. In certain embodiments, Rapamycin is initially present at a concentration of approximately 0.1 nM to approximately 500 nM, approximately 0.5 nM to approximately 250 nM, approximately 1.0 nM to approximately 150 nM, or approximately 1.5 nM to approximately 30 nM. In another embodiment, the inhibitor is LY 294002. In certain embodiments, LY 294002 is initially present at a concentration of approximately 1 μM to approximately 500 μM, approximately 2.5 μM to approximately 400 μM, approximately 5 μM to approximately 250 μM, approximately 10 μM to approximately 200 μM or approximately 20 μM to approximately 163 μM. In another embodiment, the inhibitor is Akt1-II. In certain embodiments, Akt1-II is initially present at a concentration of approximately 0.1 μM to approximately 500 μM, approximately 1 μM to approximately 250 μM, approximately 5 μM to approximately 20 μM, approximately 10 μM to approximately 100 μM, or approximately 40 μM.

The basal cell media may further comprise a fibroblast growth factor. In one embodiment for producing definitive endoderm cells, the FGF is bFGF. In embodiments bFGF is initially present at a concentration of approximately 0.1 ng/ml to approximately 100 ng/ml, approximately 0.5 ng/ml to approximately 50 ng/ml, approximately 1 ng/ml to approximately 25 ng/ml, approximately 1 ng/ml to approximately 12 ng/ml, or is initially present at a concentration of approximately 8 ng/ml.

In embodiments where liver endoderm cells are produced from definitive endoderm cells, the FGF is preferably FGF 10, at an effective concentration generally ranging from about 1 ng/ml to about 100 ng/ml, preferably about 10 ng/ml to about 90 ng/ml, preferably about 25 to about 75 ng/ml, preferably about 50 ng/ml. FGF is also included in basal media used to produce pancreatic endoderm cells, at about 1 to about 100 ng/ml, preferably about 10 to about 50 ng/ml.

In a further embodiment, the cell culture medium is a conditioned medium. The conditioned medium can be obtained from a feeder layer. It is contemplated that the feeder layer may comprise fibroblasts, and in one embodiment, comprises embryonic fibroblasts. This is particularly relevant where definitive endoderm cells are to be efficiently produced.

In certain preferred embodiments, the cell culture medium is a conditioned medium. The conditioned medium can be obtained from a feeder layer. It is contemplated where definitive endoderm cells are being produced, that the feeder layer comprises fibroblasts, and in one embodiment, comprises embryonic fibroblasts. In a preferred embodiment, the conditioned medium for produced definitive endoderm cells comprises DMEM/F-12 (50/50), approximately 20% KSR, approximately 0.1 mM NEAA, approximately 2 mM L-Glutamine, approximately 50 U/ml penicillin, approximately 50 μg/ml streptomycin, and approximately 8 ng/ml bFGF.

In still another embodiment, the cell culture medium for producing definitive endoderm cells comprises a member of the TGFβ family. In certain embodiments, the member of the TGFβ family is selected from the group consisting of Nodal, Activin A, Activin B, TGF-β, BMP2 and BMP4. In other embodiments, the member of the TGF-β family is Activin A or Nodal. In certain embodiments, Activin A is initially present at a concentration of approximately 1 ng/ml to approximately 1 mg/ml, approximately 10 ng/ml to approximately 500 ng/ml, approximately 25 ng/ml to approximately 250 ng/ml, approximately 50 ng/ml to approximately 200 ng/ml, or approximately 100 ng/ml. In other embodiments, Nodal is initially present at a concentration of approximately 100 ng/ml to approximately 5 mg/ml, approximately 500 ng/ml to approximately 2.5 mg/ml, approximately 800 ng/ml to approximately 1.5 mg/ml, or approximately 1 mg/ml.

In an a preferred embodiment related to the production of pancreatic endoderm cells, the conditioned medium preferably comprises DMEM/F-12 (50/50), with approximately 10% FCS, approximately 0.2 μm (micromolar) retinoic acid and 10-50 ng/ml Fgf10. In this embodiment, isolated definitive endoderm cells (pelleted from a centrifugation step) are resuspended in the above basal cell media and are plated preferably at about 7.5×10⁵ cells/100 mm plate on Matrigel coated tissue culture plates. Plates are incubated at 37° C./5% CO₂ and media is replaced daily. After 4 days, the media is changed to DMEM/F12, 10% FBS for 1-4 days (no retinoic acid or Fgf), whereupon the pancreatic endoderm cells may be isolated or alternatively carried through for further differentiation to pancreatic β cells, which may be used for therapy to treat patients with type I or II diabetes mellitus.

The present invention may be understood more readily by reference to the following detailed description of the preferred embodiments of the invention and the Examples included herein. However, before the present compositions and methods are disclosed and described, it is to be understood that this invention is not limited to specific nucleic acids, specific polypeptides, specific cell types, specific host cells, specific conditions, or specific methods, etc., as such may, of course, vary, and the numerous modifications and variations therein will be apparent to those skilled in the art.

As used herein, the term “endoderm” includes, but is not limited to, definitive endoderm; parietal endoderm, visceral endoderm, and mesendoderm cells. As used herein, the term “definitive endoderm” refers to early endoderm cells that have the capacity to differentiate into any or many of the endoderm cell types that are generated from the endoderm lineages in the embryo (i.e. pancreas, liver, lung, stomach, intestine and thyroid). Definitive endoderm cells are multipotent. Therefore, the use of the term “definitive endoderm” in the context of the present invention means that the cell is at least more differentiated towards an endoderm cell type than the pluripotent cell from which it is derived. Also, as used herein, producing an endoderm cell encompasses the production of a cell culture that is enriched for endoderm cells.

As used herein, “definitive endoderm” cells are characterized by the expression of specific marker transcripts such as SOX17, with the concomitant absence of marker transcripts extraembryhonic endoderm such as for AFP and thrombomodulin. In addition, such cells can express CXCR4, GATA4, GATA4.6 and GSC. Additionally, LY treatment results in the loss of a subset of cell surface CD markers initially expressed by undifferentiated hES cells, including, but not limited to, CD9, 27, 30, 46, 58 and 81. In some embodiments of the present invention, definitive endoderm cells express the SOX17 marker gene at a level higher than that of SOX7, a marker gene characteristic of visceral endoderm. Additionally, in certain embodiments, expression of the SOX17 marker gene is higher than the expression of the OCT4 marker gene, which is characteristic of hESCs. In other embodiments of the present invention, definitive endoderm cells express the SOX17 marker gene at a level higher than that of the AFP, SPARC or Thrombomodulin (TM) marker genes. In embodiments of the present invention, the definitive endoderm cells produced by the methods described herein do not express Pdx1 or Isl1 (Pdx1-negative or Isl1-negative). In another embodiment, the definitive endoderm cells display similarly low expression of thrombomodulin as seen in a population of pluripotent cells as determined, for example, by flow cytometry.

In certain embodiments of the present invention, the definitive endoderm cell cultures used are substantially free of cells expressing the OCT4, SOX7, AFP, SPARC, TM, ZIC1 or BRACH marker genes. In other embodiments, the definitive endoderm cell cultures used are substantially free of cells expressing the SOX7, AFP, SPARC, TM, ZIC1 or BRACH marker genes. With respect to cells in cell cultures, the term “substantially free of” means that the specified cell type is present in an amount of less than about 5% of the total number of cells present in the cell culture.

The term “pancreatic endoderm” refers to endoderm cells derived from definitive endoderm cells, which have been exposed to effective amounts of retinoic acid alone, or in combination with other growth factors such as fibroblast growth factor (e.g. Fgf 10) and which express the marker Pdx1 and Isl1 marker and which may be further differentiated to pancreatic β cells.

The term “liver endoderm” refers to endoderm cells derived from definitive endoderm cells, which have been exposed to growth factors such as fibroblast growth factor (e.g. Fgf 10) in the absence of retinoic acid.

As used herein, the term “differentiate” refers to the production of a cell type that is more differentiated than the cell type from which it is derived. The term therefore encompasses cell types that are partially and terminally differentiated.

In certain embodiments of the present invention, the term “enriched” refers to a cell culture that contains more than approximately 500%, 55%, 60%, 65%, 70%, 75%, 80%, 85%, 90%, or 95% of the desired cell lineage, depending upon the type of cells and methods used to provide same.

The definitive endoderm cell types produced using the present invention that differentiate from embryonic stem cells after contact with inhibitors of the PI3-kinase pathway may be used to produce pancreatic endoderm cells or liver endoderm cells according to the present invention.

Cells which are produced according to the present invention have several uses in various fields of research and development including but not limited to drug discovery, drug development and testing, toxicology, production of cells for therapeutic purposes and for transplantation as well as basic science research. These cell types express molecules that are of interest in a wide range of research fields. These include the molecules known to be required for the functioning of the various cell types as described in standard reference texts. These molecules include, but are not limited to, cytokines, growth factors, cytokine receptors, extracellular matrix, transcription factors, secreted polypeptides (hormones) and other molecules, and growth factor receptors.

In a preferred embodiment, the definitive endoderm cell is a human cell and the pancreatic endoderm and/or liver endoderm cells are human cells. These cells are derived according to the methods of the present invention using pluripotent human cells.

As used herein, the term “pluripotent human cell” or “human embryonic stem cells” encompasses pluripotent cells obtained from human embryos, fetuses or adult tissues. In one preferred embodiment, the pluripotent human cell is a human pluripotent embryonic stem cell (hESC). In another embodiment the pluripotent human cell is a human pluripotent fetal stem cell, such as a primordial germ cell. In another embodiment the pluripotent human cell is a human pluripotent adult stem cell. As used herein, the term “pluripotent” refers to a cell capable of at least developing into one of ectodermal, endodermal and mesodermal cells. As used herein the term “pluripotent” refers to cells that are totipotent and multipotent. As used herein, the term “totipotent cell” refers to a cell capable of developing into all lineages of cells. The term “multipotent” refers to a cell that is not terminally differentiated. As also used herein, the term “multipotent” refers to a cell that, without manipulation (i.e., nuclear transfer or dedifferentiation inducement), is incapable of forming differentiated cell types derived from all three germ layers (mesoderm, ectoderm and endoderm), or in other words, is a cell that is partially differentiated. The pluripotent human cell can be selected from the group consisting of a human embryonic stem (ES) cell; a human inner cell mass (ICM)/epiblast cell; a human primitive ectoderm cell, such as an early primitive ectoderm cell (EPL); a human primordial germ (EG) cell; and a human teratocarcinoma (EC) cell. The human pluripotent cells of the present invention can be derived using any method known to those of skill in the art. For example, the human pluripotent cells can be produced using de-differentiation and nuclear transfer methods. Additionally, the human ICM/epiblast cell or the primitive ectoderm cell used in the present invention can be derived in vivo or in vitro. EPL cells may be generated in adherent culture or as cell aggregates in suspension culture, as described in WO 99/53021. Furthermore, the human pluripotent cells can be passaged using any method known to those of skill in the art, including, manual passaging methods, and bulk passaging methods such as antibody selection and protease passaging.

In certain embodiment, the embryonic stem cell of the invention has a normal karyotype, while in other embodiments, the embryonic stem cell has an abnormal karyotype. In one embodiment, a majority of the embryonic stem cells have an abnormal karyotype. It is contemplated that greater than 50%, 55%, 60%, 65%, 70%, 75%, 80%, 85%, 90% or greater than 95% of metaphases examined will display an abnormal karyotype. In certain embodiments, the abnormal karyotype is evident after the cells have been cultured for greater than 5, 6, 7, 8, 9, 10, 11, 12, 13, 14, 15, or 20 passages. In one embodiment, the abnormal karyotype comprises a trisomy of at least one autosomal chromosome, wherein the autosomal chromosome is selected from the group consisting of chromosomes 1, 7, 8, 12, 14, and 17. In another embodiment, the abnormal karyotype comprises a trisomy of more than one autosomal chromosome, wherein at least one of the more than one autosomal chromosomes is selected from the group consisting of chromosomes 1, 7, 8, 12, 14, and 17. In one embodiment, the autosomal chromosome is chromosome 12 or 17. In another embodiment, the abnormal karyotype comprises an additional sex chromosome. In one embodiment, the karyotype comprises two X chromosomes and one Y chromosome. It is also contemplated that translocations of chromosomes may occur, and such translocations are encompassed within the term “abnormal karyotype.” Combinations of the foregoing chromosomal abnormalities are also encompassed by the invention.

As recited above, in certain embodiments, the invention encompasses as a first step, a method of differentiating a pluripotent mammalian cell comprising: (a) providing the pluripotent mammalian cell, and (b) contacting the pluripotent mammalian cell with an effective amount of an inhibitor of the PI3-kinase signaling pathway to at least partially differentiate the pluripotent cell to a cell of the endoderm lineage. In one embodiment, step (b) comprises the use of a cell differentiation environment. In another embodiment, the cells can be contacted with a cell differentiation environment after step (b). Additional steps according to the present invention comprise exposing definitive endoderm cells to retinoic acid in a cell differentiation environment (e.g. a basal cell media) to produce pancreatic endoderm cells. Alternatively, definitive endoderm cells may be exposed to a fibroblast growth factor (e.g. Fgf 10) in a cell differentiation environment (e.g. a basal cell media) in the absence of retinoic acid to produce liver endoderm cells.

As used herein, the term “cell differentiation environment” refers to a cell culture condition (e.g. generally, a basal cell media) wherein the pluripotent cells are induced to differentiate, or are induced to become a human cell culture enriched in differentiated cells. Preferably, the differentiated cell lineage induced by the growth factor will be homogeneous in nature. The term “homogeneous,” refers to a population that contains more than approximately 50%, 60%, 70%, 80%, 85%, 86%, 87%, 88%, 89%, 90%, 91%, 92%, 93%, 94%, 95%, 96%, 97%, 98%, or 99% of the desired cell lineage. A homogeneous lineage may be obtained directly from the differentiation process without further purification of the cells or alternatively, flow cytometry and other techniques may be used to purify the cells, especially the pancreatic endoderm cells or liver endoderm cells.

In one embodiment, the pluripotent cells are induced to differentiate into cells of the definitive endoderm lineage, which may be further differentiated to produce pancreatic endoderm cells or liver endoderm cells. Preferably, the pluripotent cells are induced to differentiate into a population of cells comprising greater than approximately 50% definitive endoderm cells. In other embodiments, the population comprises greater than approximately 55%, 60%, 65%, 70%, 75%, 80%, 85%, 90%, 91%, 92%, 93%, 94%, 95%, 96%, 97%, 98%, or 99% of the definitive endoderm lineage. The endoderm cells may be separated or used directly without separation or purification to produce pancreatic endoderm or liver endoderm cells hereunder.

A differentiating medium or environment (generally, a basal cell media) may be utilized to differentiate the pluripotent cells of the present invention, either prior to, during, or after contacting the pluripotent cells with an inhibitor of PI3-kinase. In the case of producing pancreatic endoderm cells, the differentiating agent is an effective amount of retinoic acid, which may be used prior to, during or after contacting the definitive endoderm cells with the differentiating medium (basal cell media as otherwise described herein).

In accordance with the invention the cell differentiation medium (basal cell medium) to form the definitive, pancreatic or liver endoderm cells may contain a variety of components as described hereinabove, including, for example, KODMEM medium (Knockout Dulbecco's Modified Eagle's Medium), DMEM, Ham's F12 medium (especially DMEM/F12 50:50), FBS or FCS (fetal bovine serum or fetal calf serum), fibroblast growth factor, including FGF2 (fibroblast growth factor 2), FGF 8, FGF 10 (especially for pancreatic or liver endoderm cells), KSR or hLIF (human leukemia inhibitory factor). The cell differentiation medium can also contain supplements such as L-Glutamine, NEAA (non-essential amino acids), P/S (penicillin/streptomycin), N2 and β-mercaptoethanol (β-ME). It is contemplated that additional factors may be added to the cell differentiation environment, including, but not limited to, fibronectin, laminin, heparin, heparin sulfate, retinoic acid, members of the epidermal growth factor family (EGFs), members of the fibroblast growth factor family (FGFs) including FGF2, FGF8 and/or FGF10, members of the platelet derived growth factor family (PDGFs), transforming growth factor (TGF)/bone morphogenetic protein (BMP)/growth and differentiation factor (GDF) factor family antagonists including but not limited to noggin, follistatin, chordin, gremlin, cerberus/DAN family proteins, ventropin, high dose activin, and amnionless. TGF/BMP/GDF antagonists could also be added in the form of TGF/BMP/GDF receptor-Fc chimeras. Other factors that may be added include molecules that can activate or inactivate signaling through Notch receptor family, including but not limited to proteins of the Delta-like and Jagged families as well as inhibitors of Notch processing or cleavage. Other growth factors may include members of the insulin like growth factor family (IGF), insulin, the wingless related (WNT) factor family, and the hedgehog factor family. Additional factors may be added to promote definitive endoderm stem/progenitor proliferation and survival as well as survival and differentiation of derivatives of these progenitors.

In other embodiments, the methods comprises plating the cells in an adherent culture. As used herein, the terms “plated” and “plating” refer to any process that allows a cell to be grown in adherent culture. As used herein, the term “adherent culture” refers to a cell culture system whereby cells are cultured on a solid surface, which may in turn be coated with a solid substrate that may in turn be coated with another surface coat of a substrate, such as those listed below, or any other chemical or biological material that allows the cells to proliferate or be stabilized in culture. The cells may or may not tightly adhere to the solid surface or to the substrate. In one embodiment, the cells are plated on matrigel coated plates, which is preferred. The substrate for the adherent culture may comprise any one or combination of polyornithine, laminin, poly-lysine, purified collagen, gelatin, extracellular matrix, fibronectin, tenascin, vitronectin, entactin, heparin sulfate proteoglycans, poly glycolytic acid (PGA), poly lactic acid (PLA), poly lactic-glycolic acid (PLGA) and feeder layers such as, but not limited to, primary fibroblasts or fibroblast cells lines. Furthermore, the substrate for the adherent culture may comprise the extracellular matrix laid down by a feeder layer, laid down by the pluripotent human cells or cell culture or laid down by the definitive endoderm cells or cell culture.

The methods of the present invention contemplate that cells may be cultured with a feeder cell or feeder layer. The term “feeder cell” is used to describe a cell that is co-cultured with a target cell and stabilizes the target cell in its current state of differentiation. A feeder layer comprises more than one feeder cell in culture. In one embodiment of the above method, conditioned medium is obtained from a feeder cell that stabilizes the target cell in its current state of differentiation. Any and all factors produced by a feeder cell that allow a target cell to be stabilized in its current state of differentiation can be isolated and characterized using methods routine to those of skill in the art. These factors may be used in lieu of a feeder layer, or may be used to supplement a feeder layer.

As used herein, the term “stabilize” refers to the differentiation state of a cell. When a cell or cell population is stabilized, it will continue to proliferate over multiple passages in culture, and preferably indefinitely in culture; additionally, each cell in the culture is preferably of the same differentiation state, and when the cells divide, typically yield cells of the same cell type or yield cells of the same differentiation state. Preferably, a stabilized cell or cell population does not further differentiate or de-differentiate if the cell culture conditions are not altered, and the cells continue to be passaged and are not overgrown. Preferably the cell that is stabilized is capable of proliferation in the stable state indefinitely, or for at least more than 2 passages. Preferably, it is stable for more than 5 passages, more than 10 passages, more than 15 passages, more than 20 passages, more than 25 passages, or most preferably, it is stable for more than 30 passages. In certain embodiments, the cell is stable for greater than 1 year of continuous passaging.

In one embodiment, stem cells (pluripotent cells) to be differentiated into definitive endoderm cells are maintained in culture in a pluripotent state by routine passage until it is desired that they be differentiated into definitive endoderm. In some embodiments, a member of the TGFβ family is administered to the pluripotent cell in conjunction with the inhibitor of the PI3-kinase pathway. As used herein, the term “member of the TGF-β family” refers to growth factors that are generally characterized by one of skill in the art as belonging to the TGF-β family, either due to homology with known members of the TGF-β family, or due to similarity in function with known members of the TGF-β family. In certain embodiments, the member of the TGF-β family is selected from the group consisting of Nodal, Activin A, Activin B. TGF-β, BMP2 and BMP4. In one embodiment, the member of the TGF-β family is Activin A. Additionally, the growth factor Wnt3a is useful for the production of definitive endoderm cells. In certain embodiments of the present invention, combinations of any of the above-mentioned growth factors can be used. It is not necessary that these components be added to the cells simultaneously.

In at least one embodiment, definitive endoderm cells are maintained in culture by routine passage until it is desired that they be differentiated into pancreatic endoderm or liver endoderm. In some embodiments, a member of the FGF family (e.g., preferably FGF 10) is administered to the definitive endoderrn cell in conjunction with the retinoic acid differentiation agent to produce pancreatic endoderm or liver endoderm cells.

With respect to some of the embodiments of differentiation methods described herein, the above-mentioned growth factors are provided to the cells so that the growth factors are present in the cultures at concentrations sufficient to promote differentiation of at least a portion of the stem cells to definitive endoderm and/or definitive endoderm cells to pancreatic endoderm cells or liver endoderm cells. In some embodiments of the present invention, the above-mentioned growth factors are present in the cell culture at a concentration of at least about 0.5 ng/ml, at least 1 ng/ml, at least 10 ng/ml, at least about 25 ng/ml, at least about 50 ng/ml, at least about 75 ng/ml, at least about 100 ng/ml, at least about 200 ng/ml, at least about 300 ng/ml, at least about 400 ng/ml, at least about 500 ng/ml, or at least about 1000 ng/ml.

In certain embodiments of the present invention, the above-mentioned growth factors are removed from the cell culture subsequent to their addition. For example, the growth factors can be removed within about one day, about two days, about three days, about four days, about five days, about six days, about seven days, about eight days, about nine days or about ten days after their addition. In a preferred embodiment, the growth factors are removed about four days after their addition.

Cultures of definitive endoderm cells, pancreatic endoderm cells or liver endoderm cells can be grown in medium containing reduced serum or no serum. In certain embodiments of the present invention, serum concentrations can range from about 0.1% to about 20% (v/v). In some embodiments, definitive endoderm cells are grown with serum replacement. In other embodiments, definitive endoderm cells are grown in the presence of B27. In such embodiments, the concentration of B27 supplement can range from about 0.2% to about 20% (v/v) or greater than about 20% (v/v). Alternatively, the concentration of the added B27 supplement can be measured in terms of multiples of the strength of a commercially available B27 stock solution. For example, B27 is available from Invitrogen (Carlsbad, Calif.) as a 50× stock solution. Addition of a sufficient amount of this stock solution to a sufficient volume of growth medium produces a medium supplemented with the desired amount of B27. For example, the addition of 10 ml of 50× B27 stock solution to 90 ml of growth medium would produce a growth medium supplemented with 5× B27. The concentration of B27 supplement in the medium can be about 0.1×, about 0.2×, about 0.3×, about 0.4×, about 0.5×, about 0.6×, about 0.7×, about 0.8×, about 0.9×, about 1×, about 1.1×, about 1.2×, about 1.3×, about 1.4×, about 1.5×, about 1.6×, about 1.7×, about 1.8×, about 1.9×, about 2×, about 2.5×, about 3×, about 3.5×, about 4×, about 4.5×, about 5×, about 6×, about 7×, about 8×, about 9×, about 10×, about 11×, about 12×, about 13×, about 14×, about 15×, about 16×, about 17×, about 18×, about 19×, about 20× and greater than about 20×. In preferred embodiments, both pancreatic endoderm cells and liver endoderm cells are preferably grown in basal cell media comprising about 1% to about 20% (vol.) fetal calf serum, more preferably about 10% fetal calf serum.

The progression of the hESC culture to definitive endoderm or from definitive endoderm to pancreatic endoderm or liver endoderm can be monitored by quantitating expression of marker genes characteristic of these cells as well as the lack of expression of marker genes characteristic of hESCs, definitive endoderm cells (in the case of pancreatic or liver endoderm cells) and other cell types. One method of quantitating gene expression of such marker genes is through the use of quantitative PCR (Q-PCR). Methods of performing Q-PCR are well known in the art. Other methods which are known in the art can also be used to quantitate marker gene expression. Marker gene expression can be detected by using antibodies specific for the marker gene of interest.

Using the methods described herein, compositions comprising definitive endoderm cells, pancreatic endoderm cells or liver endoderm cells which are substantially free of other cell types can be produced. Alternatively, compositions comprising mixtures of hESCs and definitive endoderm, or definitive endoderm cells and pancreatic endoderm cells or liver endoderm cells can be produced. For example, compositions comprising at least 5 definitive endoderm cells for every 95 hESCs can be produced, or 5 pancreatic endoderm cells or liver endoderm cells for every 95 definitive endoderm cells can be produced. In still other embodiments, compositions comprising at least 95 definitive endoderm cells for every 5 hESCs, or up to 80 or more pancreatic or live endoderm cells for every 5 definitive endoderm cells can be produced. Additionally, compositions comprising other ratios of definitive endoderm cells to hESCs or pancreatic endoderm or liver endoderm cells to definitive endoderm cells are contemplated.

In some embodiments of the present invention, definitive endoderm cells, pancreatic endoderm cells or liver endoderm cells can be isolated by using an affinity tag that is specific for such cells. One example of an affinity tag specific for definitive endoderm cells, pancreatic endoderm cells or liver endoderm cells is an antibody that is specific to a marker polypeptide that is present on the cell surface of the endoderm cells desired to be purified but which is not substantially present on other cell types that would be found in a cell culture produced by the methods described herein.

It is contemplated that the pluripotent cells or definitive endoderm cells which are used as starting materials can be dissociated to an essentially single cell culture prior to being contacted with the inhibitor of the PI3-kinase signaling pathway or with retinoic acid (optionally including an FGF such as FGF 10) to produce pancreatic endoderm cells or an FGF (e.g. FGF 10) in the absence of retinoic acid to produce liver endoderm cells. As used herein, an “essentially single cell culture” is a cell culture wherein during passaging, the cells desired to be grown are dissociated from one another, such that the majority of the cells are single cells, or two cells that remain associated (doublets). Preferably, greater than 40%, 50%, 60%, 70%, 80%, 90%, 95%, 96%, 97%, 98%, 99% or more of the cells desired to be cultured are singlets or doublets. The term encompasses the use of any method known now or later developed that is capable of producing an essentially single cell culture. Non-limiting examples of such methods include the use of a cell dispersal buffer, and the use of proteases such as trypsin, collagenase, dispase, and accutase. These proteases and combinations of certain of the proteases are commercially available. The invention contemplates that the cell culture can be dissociated to an essentially single cell culture at any point during passaging, and it is not necessary that the dissociation occur during the passage immediately prior to contact with the inhibitor. The dissociation can occur during one or more passages. Alternatively, the samples may be centrifuged to dissociate the cell culture.

The cells produced using the methods of the present invention have a variety of uses. In particular, the cells can be used as a source of nuclear material for nuclear transfer techniques and used to produce cells, tissues or components of organs for transplant. For example, if a pancreatic endoderm cell or a liver endoderm cell is produced, it can be used in human cell therapy or human gene therapy to treat diseases such as type 1 diabetes, liver diseases and any other diseases that affect the pancreas or liver. In one embodiment of the foregoing, the pancreatic endoderm cell is used to treat diabetes or is further differentiated to produce pancreatic β cells for use in the treatment of diabetes. In addition, the cells may be used for toxicity or drug screens.

Throughout this application, various publications are referenced. The disclosures of all of these publications and those references cited within those publications in their entireties are hereby incorporated by reference into this application in order to more fully describe the state of the art to which this invention pertains.

### EXAMPLES

### Example 1

### Culture of Human ES Cells

### Routine Human ES Cell Culture

The human embryonic stem cell line BG01 (BresaGen, Inc., Athens, Ga.) may be used in this work. BG01 cells are grown in hES Medium, consisting of DMEM/F-12 (50/50) supplemented with 20% knockout serum replacer (KSR; Invitrogen), 0.1 mM MEM Non-essential amino acids (NEAA; Invitrogen), 2 mM L-Glutamine (Invitrogen), 50 U/ml penicillin, 50 μg/ml streptomycin (Invitrogen), 4 ng/ml bFGF (Sigma) and 0.1 mM β-mercaptoethanol (Sigma). The cells are grown on mouse primary embryonic fibroblast feeder layers that were mitotically inactivated with mitomycin C. Feeder cells are plated at 1.2×10⁶ cells per 35 mm dish. The BG01 cells are passaged using a collagenase/trypsin method. Briefly, medium is removed from the dish, 1 ml of 200 U/ml Collagenase type IV (GibcoBRL) is added, and the cells are incubated at 37° C. for 1-2 minutes. Collagenase is removed and 1 ml of 0.05% trypsin/0.53 mM EDTA (GIBCO) is applied. Cells are incubated at 37° C. for 30 seconds and then washed from the feeder layer, and the trypsin is inactivated in DMEM/F-12 with 10% fetal bovine serum (FBS; Hyclone). Cells are replated on feeder layers at 100,000-300,000 cells per 35 mm dish and are passaged every 3 days.

### Growth of BG01 Cells in Feeder Free Conditions

hES medium (25 mls) is conditioned overnight on mitomycin treated MEFs plated in 75 cm² flasks at 56,000 cells/cm². The MEFs are used for up to 1 week with conditioned medium (CM) collection every 24 hours. CM is supplemented with an additional 8 ng/ml of hbFGF before use. Matrigel coated dishes are prepared by diluting Growth Factor Reduced BD matrigel matrix (BD Biosciences) to a final concentration of 1:30 in cold DMEM/F-12. 1 ml/35 mm dish is used to coat dishes for 1-2 hours at room temperature or at least overnight at 4° C. Plates were stored up to one week at 4° C. Matrigel solution is removed immediately before use.

### Embryoid Body Formation

The BG01 cells are disaggregated using the Collagenase/trypsin method described above. Approximately 10,000 cells are suspended in 50 μl of EB medium (DMEM (Celigro) supplemented with 10% FBS (Atlanta Biolabs), 0.1 mM NEAA, 2 mM L-Glutamine, 50 U/ml penicillin and 50 μg/ml streptomycin), and are dropped onto a 100 mm Petri dish lid with a p200 pipette tip. Approximately 50 drops are placed per lid. The lid is placed onto the dish and 10 ml of PBS is placed in the dish. EBs are washed from the lid at 3 and 5 days, incubated with trypsin for 5 minutes at room temperature and disaggregated with a drawn out glass pipette. Cells are washed once in 1×PBS and fixed in 2% PFA/2% sucrose for 10 minutes at room temperature. Cells are then washed twice in PBS and stored in 1% BSA/PBS ready for antibody staining.

### Example 2

### Treatment of HES Cells with Inhibitors of PI3-Kinase Leads to Differentiation of the HES Cells

### Inhibitor/Differentiation Agent Treatment of Stem Cells

BG01 cells are passaged from feeders using the collagenase/trypsin method and are plated on matrigel coated dishes at 1×10⁵ cells/35 mm dish in conditioned medium (CM; MEF conditioned medium plus 8 ng/ml bFGF). After approximately 24 hours, the media is replaced with fresh CM, CM with inhibitor (resuspended in EtOH), CM with EtOH, or with Spontaneous Differentiation medium (hES medium minus bFGF).

In alternative methods, the BG01 cells may be plated at different concentrations prior to contact with CM, CM with inhibitor and CM with EtOH Cells may be plated at the following concentrations: approximately 5×10⁴ cells/35 mm dish, approximately 1×10⁵ cells/35 mm dish, approximately 2×10⁵ cells/35 mm dish, approximately 4×10⁵ cells/35 mm dish and, approximately 6×10⁵ cells/35 mm dish.

The inhibitor LY 294002 (Biomol) may be preferably used at the concentration range approximately 20-163 μM and the inhibitor Rapamycin (Calbiochem) is used at the concentration range approximately 1.5-30 nM. LY 294002 inhibits the PI3-kinase pathway by binding to the ATP docking site of p110. Rapamycin inhibits a subset of the PI3-kinase pathway by inhibiting mTOR (mammalian target of rapamycin).

Cells are grown in these conditions for approximately 72 hours with a medium change every 24 hours. Cells are harvested using the collagenase/trypsin method for flow cytometry and RT-PCR analysis and are scraped for biochemical analysis.

By observing the cells using standard microscopy, it is noted that BG01 cells undergo morphological change when cultured in the presence of either LY 294002 or rapamycin. This morphological change is notably different from the change in cells undergoing spontaneous differentiation. In undifferentiated cultures, individual cells are not easily discernable, being relatively small, irregular and without clearly apparent intercellular junctions at higher density. After treatment with LY 294002, however, the cells underwent marked spreading and adopted obvious epitheloid cuboidal morphologies. Individual cells are also more readily discernable at higher densities since discrete intercellular adhering junctions are evident between neighboring cells.

Additionally, cells plated at concentrations lower than approximately 2×10⁵ cells/35 mm dish display changes in morphology when contacted with LY 294002 or rapamycin. Cells plated at densities of approximately 4×10⁵ cells/35 mm dish or higher do not demonstrate the same morphological changes upon contact with LY 294002 or rapamycin.

### Example 3

### Characteristics of Cells Treated with Inhibitors of PI3-Kinase

The inhibitor studies are performed as described in Example 2.

### Flow Cytometry

For flow cytometry, the BG01 cells are washed with 1×PBS and fixed in 2% paraformaldehyde/1×PBS for 10 minutes at room temperature. The cells are then washed in 1×PBS and approximately 2×10⁵ cells are incubated with primary antibody diluted in 1% BSA/1×PBS. The primary antibodies used are anti-CD9 and anti-thrombomodulin (Cymbus Biotechnology), FITC conjugated mouse monoclonal antibodies at a 1:10 dilution. Cells are incubated at 4° C. for 30 minutes and then washed twice in 1×PBS. Where appropriate, cells are resuspended in a secondary antibody, anti-mouse Alexa-488 (Molecular Probes) diluted 1:1000 in 1% BSA/PBS, incubated at 4° C. for 30 minutes, and then washed twice in 1×PBS. Cells are resuspended in 1% BSA/1×PBS and surface expression is analyzed using a Beckman Coulter FC500.

### RNA Isolation and RT-PCR Analysis

Total RNA is isolated using TRIzol Reagent (GibcoBRL). RNA is run on a 1% agarose gel containing ethidium bromide and visualized using the AlphaImager™ 2200 Documentation and Analysis System to ensure RNA integrity. 10 μg of RNA is treated with DNase (Ambion), which is removed with DNase Inactivation Reagent (Ambion). cDNA is prepared with 500 ng of total RNA using the Superscript II Reverse transcriptase Kit (Invitrogen) using oligo(dT) primers. PCR reactions are carried out on 1 μl of cDNA. PCR products are run on a 2% agarose gel containing ethidium bromide and visualized using the Alphaamager™ 2200 Documentation and Analysis System. PCR primer sets used were GATA4, Mix1, Msx1, AFP, HNF4alpha, eHAND, Nanog, AFP and GAPDH.

### Biochemical Analysis

Activity of the PI3-kinase signaling pathway may be assessed by Western blot analysis. Briefly, detergent extracts are prepared from untreated and treated cell cultures, separated by SDS-PAGE and blotted to nitrocellulose. Expression of active forms of the PI3-kinase intracellular targets PKB/Akt, S6 kinase and S6 protein are then determined by probing the nitrocellulose with appropriate antibodies to phosphorylated forms of each of these proteins. Generally, 30 μg of total protein are evaluated for each sample, primary incubations were carried out at a 1:1000 dilution of antibody, and binding in each case is detected by standard ECL based methodology.

### Q-PCR Gene Expression Assay

Real-time measurements of gene expression are analyzed for multiple marker genes at multiple time points by Q-PCR. Marker genes characteristic of the desired as well as undesired cell types are evaluated to gain a better understanding of the overall dynamics of the cellular populations. The strength of Q-PCR analysis includes its extreme sensitivity and relative ease of developing the necessary markers, as the genome sequence is readily available. Furthermore, the extremely high sensitivity of Q-PCR permits detection of gene expression from a relatively small number of cells within a much larger population. In addition, the ability to detect very low levels of gene expression may provide indications for “differentiation bias” within the population. The bias towards a particular differentiation pathway, prior to the overt differentiation of those cellular phenotypes, would likely be unrecognizable using immunocytochemical techniques. For this reason, Q-PCR provides a method of analysis that is complementary to immunocytochemical techniques for screening the success of differentiation treatments. This tool provides a means of evaluating our differentiation protocol successes in a quantitative format at semi-high throughput scales of analysis.

The general approach is to perform relative quantitation using SYBR Green chemistry on the Rotor Gene 3000 instrument (Corbeft Research) and a two-step RT-PCR format. Primers are designed to lie over exon-exon boundaries or span introns of at least 800 bp when possible, as this eliminates amplification from contaminating genomic DNA. When marker genes are employed that do not contain introns or they possess pseudogenes, DNase I treatment of RNA samples may be performed. The markers relevant for the early phases of hESC differentiation (specifically ectoderm, mesoderm, definitive endoderm and extra-embryonic endoderm) and for which validated primer sets exist are provided below in Table 1. The human specificity of these primer sets has also been demonstrated. This is an important fact since the hESCs are often grown on mouse feeder layers. Typically, triplicate samples are taken for each condition and independently analyzed in duplicate to assess the biological variability associated with each quantitative determination.

Total RNA is isolated using RNeasy (Qiagen) and quantitated using RiboGreen (Molecular Probes). Reverse transcription from 350-500 ng of total RNA is carried out using the iScript reverse transcriptase kit (BioRad), which contains a mix of oligo-dT and random primers. Each 20 μL reaction is subsequently diluted up to 100 μL total volume and 3 μL is used in each 10 μL Q-PCR reaction containing 400 nM forward and reverse primers and 5 μL 2×SYBR Green master mix (Qiagen). Two step cycling parameters is used employing a 5 second denature at 85-94° C. (specifically selected according to the melting temp of the amplicon for each primer set) followed by a 45 second anneal/extend at 60° C. Fluorescence data is collected during the last 15 seconds of each extension phase. A three point, 10-fold dilution series is used to generate the standard curve for each run and cycle thresholds (Ct's) were converted to quantitative values based on this standard curve. The quantitated values for each sample are normalized to housekeeping gene performance and then average and standard deviations are calculated for triplicate samples. At the conclusion of PCR cycling, a melt curve analysis is performed to ascertain the specificity of the reaction. A single specific product is indicated by a single peak at the Tₘ appropriate for that PCR amplicon. In addition, reactions performed without reverse transcriptase may serve as the negative control and do not amplify.

Both Cyclophilin G and GUS may be used to calculate a normalization factor for all samples. The use of multiple HGs simultaneously reduces the variability inherent to the normalization process and increases the reliability of the relative gene expression values (Vandesompele, et al., 2002, Genome Biol., 3:RESEARCH0034).

Q-PCR is used to determine the relative gene expression levels of many marker genes across samples receiving different experimental treatments. The marker genes are employed because they exhibit enrichment in specific populations representative of the early germ layers and in particular have focused on sets of genes that are differentially expressed in definitive endoderm cells. These genes as well as their relative enrichment profiles are highlighted in Table 1. They assist in isolation as well as characterizing the formation of definitive endoderm, pancreatic endoderm or liver endoderm cells.

TABLE 1

| Germ Layer | Gene | Expression Domains |
|---|---|---|
| Endoderm | SOX17 | definitive, visceral and parietal endoderm |
| | MIXL1 | endoderm and mesoderm |
| | GATA4 | definitive and primitive endoderm |
| | HNF3b | definitive endoderm and primitive endoderm, |
| | | mesoderm, neural plate |
| | GSC | mesendoderm and definitive endoderm |
| | Cerebrus | primitive and definitive endoderm |
| Extra- | SOX7 | visceral endoderm |
| embryonic | AFP | visceral endoderm, liver |
| | SPARC | parietal endoderm |
| | TM | parietal endoderm/trophectoderm |
| | NODAL | Epiblast and anterior visceralendoderm |
| Ectoderm | ZIC1 | neural tube, neural progenitors |
| | SOX1 | neural progenitors |
| Mesoderm | BRACH | nascent mesoderm |
| | FOXF1 | |

### Immunohistochemistry

### SOX17 Antibody

SOX17 is expressed throughout the definitive endoderm as it forms during gastrulation and its expression is maintained in the gut tube (although levels of expression vary along the A-P axis) until around the onset of organogenesis. SOX17 is also expressed in a subset of extra-embryonic endoderm cells. No expression of this protein has been observed in mesoderm or ectoderm. As such, SOX17 is an appropriate marker for the definitive endoderm lineage when used in conjunction with markers to exclude extra-embryonic lineages.

A SOX17 antibody may be generated as described in U.S. Provisional Application No. 60/532,004, filed Dec. 23, 2003, entitled “Definitive Endoderm”, hereby incorporated by reference in its entirety. Briefly, a portion of the human SOX17 cDNA corresponding to amino acids 172414 in the carboxyterminal end of the SOX17 protein is used for production of SOX17 antibody by genetic immunization in rats at the antibody production company, GENOVAC (Freiberg, Germany), according to procedures developed there. Procedures for genetic immunization can be found in U.S. Pat. Nos. 5,830,876, 5,817,637, 6,165,993, 6,261,281 and International Publication No. WO99/13915, the disclosures of which are incorporated herein by reference in their entireties. The antibody is determined to be specific for SOX17 by both Western blot and ICC on hSOX17 cDNA transfected cell lines.

Cells to be immunostained may be grown on chamber slides, and are rinsed once with 1×PBS and fixed for 10 minutes in 4% PFA/4% sucrose in PBS pH 7.4 at room temperature. They are then rinsed 3× in 1×PBS and blocked in 3% goat serum with 0.1% Triton-X100 in PBS for 1 hour at room temperature. Primary antibodies are diluted in 3% goat serum in PBS and this solution is applied overnight at 4° C. The primary antibodies used are rabbit anti-human AFP (Zymed), used at a 1:50 dilution, and rat anti-human SOX17 (obtained from Cythera, Inc.), used at 1:1000 dilution. Cells are washed for 1 hour with 3 changes of 1×PBS. Secondary antibodies are applied for 2 hours at room temperature. Secondary antibodies which may be used are goat anti-rabbit Alexa Fluor 488 and goat anti-rat Alexa Fluor 594 (Molecular Probes), both at a 1:1000 dilution in 3% goat serum in 1×PBS. Cells are washed for 1 hour with 3 changes of 1×PBS. The chambers are removed and slides are mounted in VectaShield mounting medium with DAPI (Vector).

### Results

Using flow cytometry, it is noted that expression of CD9 may decrease more rapidly in LY 294002 or rapamycin treated BG01 cells than in spontaneous differentiation in adherent culture, or in embryoid bodies. In addition, expression of CD9 has been previously observed by others to influence cell proliferation, motility and adhesion.

By RT-PCR analysis, it is noted that a number of markers indicative of early differentiation may be detected in cells treated with LY 294002 and rapamycin. Notably, the markers which are detected when PI3-kinase signaling is blocked may differ from those detected in spontaneously differentiating adherent cultures of BG01 cells. For example, blocking PI3-kinase may result in an increase in levels of HNF4alpha, GATA4, Mix1, and Msx1, and a decrease in levels of AFP in comparison to spontaneously differentiating cultures.

Additionally, the differences in cell morphology that may be noted with varying densities of pluripotent cells are supported by PCR data. The effect of treatment with LY 294002 or Rapamycin may in some circumstances be dependent on cell density.

By biochemical analysis, it is noted that the activity of Akt, S6 kinase and S6 is inhibited in cells maintained in the presence of LY 294002. Similarly, the activity of S6kinase and S6 is abolished in cells maintained in the presence of rapamycin.

Collectively the observations indicate that PI3-kinase signaling to mTOR is down-regulated in BG01 cells in the presence of these inhibitory drugs. The activity of S6 (a distal target in this signaling pathway) is diminished in BG01 cells undergoing spontaneous differentiation in adherent culture, but was abolished in inhibitor treated cells.

### Example 4

### Preparation of Pancreatic Endoderm Cells from Human Embryonic Stem Cells

### Methods

### Matrigel Culture:

Human ES cells from a mef feeder plate, which is 60-90% confluent, are washed 1× with PBS and 5 ml of 200400 U/ml of collagenase IV in DMEM/F12 is added per 100 mm tissue culture plate. Plates are incubated at 37° C./5% CO₂ for 30-120 minutes, until colonies begin to dislodge from the plate surface. Cells are collected by gentle trituration, placed in a 15 ml conical tube and centrifuged at 200×g for 5 minutes. Media is aspirated from the pellet and the pellet is resuspended in 10 ml 20% KSR conditioned media (CM20K) with 8 ng/ml bFGF. The cells are plated on previously prepared Matrigel:plates (1:30 dilution Matrigel in DMEM/F12) at a 1:1 to 1:6 dilution in CM20K with 8 ng/ml bFGF.

### Definitive Endoderm Differentiation:

Human ES cells cultured on Matrigel, are washed 2× with PBS. 2-3 ml Trypsin/EDTA solution is added and cells are triturated and collected in a 15 ml conical tube. DMEM/F12 with 10% FBS is added to stop the trypsinization and the cells are centrifuged at 200×g for 5 minutes. The pellet, now mostly single cells with a small population of 2-4 cell clusters, is resuspended in CM20K with 8 ng/ml bFGF and plated at 4.5×10⁵ cells/100 mm plate on Matrigel coated tissue culture plates. Plates are incubated at 37° C./5% CO₂ 16-24 hours. After this incubation the media is replaced with CM20K with 8 ng/ml bFGF, 50 ng/ml ActivinA, and 25 ug/ml LY294002. Media is replaced daily for 4-6 days.

### Pancreatic Endoderm Differentiation:

Definitive endoderm cells (from treatment above) are washed 2× with PBS. 2-3 ml Trypsin/EDTA solution is added and cells are triturated and collected in a 15 ml conical tube. DMEM/F12 with 10% FBS is added to stop the trypsinization and the cells are centrifuged at 200×g for 5 minutes. The pellet, now mostly single cells with a small population of 2-4 cell clusters, is resuspended in DMEM/F12, 10% FBS, 2 uM all-trans retinoic acid, and 10-50 ng/ml FGF10. Cells are plated at 7.5×10⁵ cells/100 mm plate on Matrigel coated tissue culture plates. Plates are incubated at 37° C./5% CO₂ and media is replaced daily. After 4 days, the media is changed to DMEM/F12, 10% FBS for 1-4 days.

In the case of liver endoderm differentiation, if the above conditions for pancreatic endoderm differentiation is followed, except that retinoic acid is excluded and the amount of FGF 10 is increased (replacing the excluded retinoic acid), liver endoderm is produced.

### Further Examples

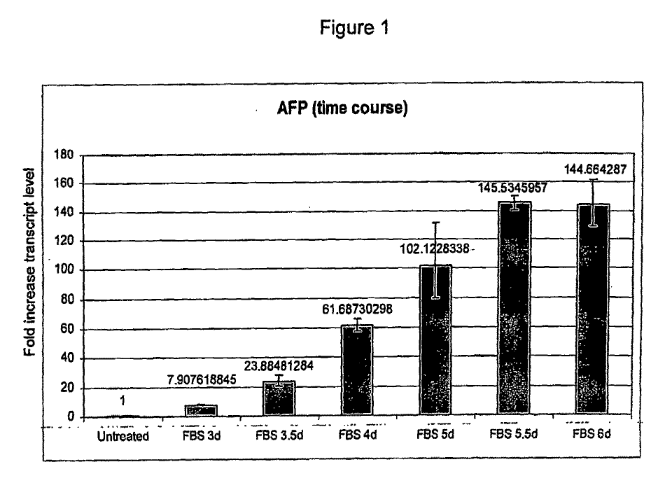

The first example relates to the generation of cells expressing the embryonic liver marker alphafetoprotein (AFP) following treatment of definitive endoderm. BG01 hESCs were differentiated into definitive endoderm for four days following the addition of LY 294002 (50 μM). Media was changed to DMEM/F12, 10% FCS and cells grown for up to six more days. Untreated: untreated hESCs. AFP transcript levels were analyzed by QRT-PCR in triplicate and shown as the fold-increase over untreated samples (hESCs) after normalization to GAPDH reference transcript. Note: this result can be achieved in the presence of absence of Fgf10 although optimal AFP induction is seen following addition of Fgf10. The results of this experiment are shown in FIG. 1.

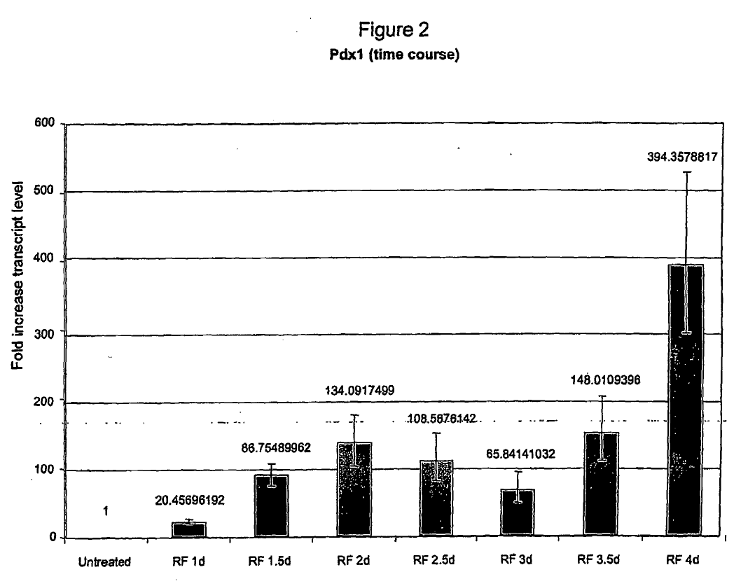

The second example looked at the time course of Pdx1 transcript induction following RA treatment. BG01 hESCs were treated with LY 294002 (50 μM) for four days then switched to media containing DMEM/F12, 10% FCS, 50 ng/ml Fgf10 and 2 μM retinoic acid for up to four days. Untreated-untreated hESCs. transcript levels were analyzed by QRT-PCR in triplicate and shown as the fold-increase over untreated samples (hESCs) after normalization to GAPDH reference transcript. Fold-induction of Pdx1 transcript levels are indicated. The results of this experiment are shown in FIG. 2.

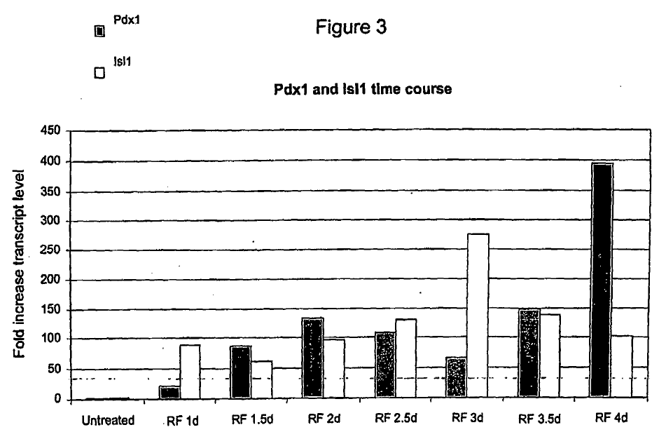

The third example looked at the time course of Pdx1 and Isl1 transcript induction following RA treatment. BG01 hESCs were treated with LY 294002 (50 μM) for four days then switched to media consisting of DMEM/F12, 10% FCS, 50 ng/ml Fgf10 and 2 μM retinoic acid for up to four days. Untreated: untreated hESCs. Transcript levels were analyzed by QRT-PCR in triplicate and shown as the fold-increase over untreated samples (hESCs) after normalization to GAPDH reference transcript. The results of this experiment are shown in FIG. 3.

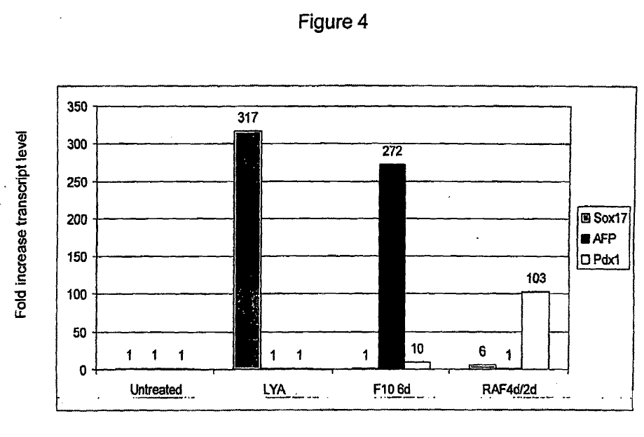

The fourth example looked at the changes in Sox17, AFP and Pdx1 in response to different culture conditions. Untreated: untreated BG01 hESCs cultured on MatriGel in the presence of MEF-CM, Fgf2, 20% KSR. LYA: hESCs grown on Matrigel in MEF-CM and Fgf2 were treated with LY 294002 for four days. F106d: hESCs treated with LY 294002 for four days were switched to media containing Fgf10 (50 ng/ml), 10% FCS for a further six days. RA4d/2d: hESCs treated with LY 294002 for 4 days were switched to media (DMEM/F12) containing Fgf10 (50 ng/ml), 2 μM RA, 10% FCS for a further four days. This was followed by culturing for a further two days in the same media lacking RA and Fgf10. Sox17, AFP and Pdx1 transcripts were analyzed by QRT-PCR in triplicate and shown as the fold-increase over untreated samples (hESCs) after normalization to GAPDH reference transcript. FIG. 4 shows the changes in Sox17, AFP and Pdx1 in response to the different culture conditions

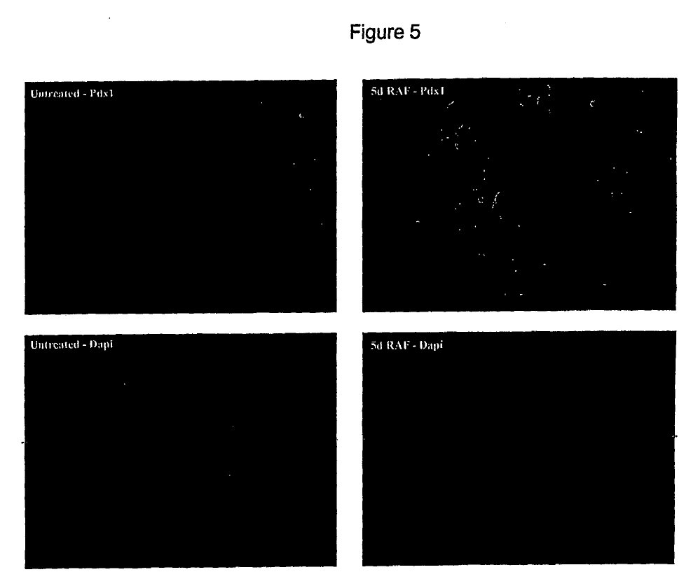

The fifth example shows the levels of Pdx1+ cells after treatment RA. In this experiment, BG01 hESCs were differentiated into definitive endoderm for four days following the addition of LY 294002 (50 μM). Media was changed to DMEM/F12, 10% FCS and cells grown for up to five more days as follows. hESCs treated with LY 294002 for 4 days were switched to media (DMEM/F12) containing Fgf10 (50 ng/ml), 2 μM RA, 10% FCS for a further five days. Treated and untreated (hESCs) were grown in LabTec chamber slides, fixed with 4% paraformaldehyde and probed a rabbit anti-human Pdx1 antibody (Chemicon, 1:1,000) followed by AlexaFluor (594 nm) labeled goat anti-rabbit secondary antibody (red). Cells were mounted in media containing DAPI for visualization of nuclear DNA (blue). FIG. 5 shows the immunofluorescence staining of Pdx1+ cells treated with RA.

Culture of hESCs:
Methods: hESCs are preferably grown in MEF-CM (details from previous filings) or defined media using Matrigel (1:200 dilution) as a growth matrix (for example). Cells are passaged manually or by using enzymatic methods such as collagenase or accutase and typically plated at 1×10⁶ per 60 mm dish for 12-24 hours in hESC media then media is changes to promote DE differentiation (see ‘Differentiation media’ below). During differentiation, ‘differentiation media’ is replaced every day.
Two examples of defined media for hESC culture are described below:
(a) DMEM:F12 (Gibco), 2% BSA (Seriologicals, #82-047-3), 1× Pen/Strep (Gibco), 1× non-essential amino acids (Gibco), 1× Trace Elements A, B and C (Cellgro; #99-182—C1, #99-176-C1, #99-175-C1), 50 ug/ml Ascorbic Acid (Sigma, #A4034), 10 ug/ml Transferrin (Gibco, ##11107-018), 0.1 nM beta-mercaptoethanol, 8 ng/ml Fgf2 (Sigma, #F0291), 200 ng/ml LR-IGF (JRH Biosciences, #85580), 10 ng/ml Activin A (R&D Systems, #338-AC), 10 ng/ml Heregulin beta (Peprotech; #100-03).
(b) A second defined media composed of; DMEM/F12, 1× Pen/Strep, 1× non essential amino acids (Gibco), Fgf2 (10 ng/ml), IGF1 (or LR-IGF; 10 ng/ml), Activin A (10 ng/ml), bovine serum albumin (fraction V or similar), transferrin (10 μg/ml, Gibco), 1× trace elements (Cellgro), β-mercaptoethanol.

Alternative defined media are described in Ludwig et al., 2006; Nature Biotech, 249(2), 185-187, 2006.

Cells grown under feeder free conditions are differentiated by changing into a defined media (no fetal calf serum or KSR-type serum replacements) containing elevated Activin A, nodal, TNFβ or other factor from the TNF family (in amounts of at least about 5 ng/ml, at least about 5-10 ng/ml, at least about 15 ng/ml, at least about 25 ng/ml, at least about 50 ng/ml, about 50-100 ng/ml) that does not support high PI3K signaling. Under normal circumstances, factors are required to promote self-renewal of hESCs by promoting PI3K activity. We have shown previously (McLean et al., 2007) that inhibition of PI3K by reducing KSR or FCS or, by addition of inhibitors provides conditions where Activin A, nodal, TNFβ or other component can promote DE differentiation. For example, the differentiation media should not have high levels of insulin, IGF or EGF family members that promote PI3K signaling (such as heregulin).

Example of such a DE differentiation media are:
(a) DMEM:F12 (Gibco), 2% BSA (Seriologicals, #82-047-3), 1× Pen/Strep (Gibco), 1× non-essential amino acids (Gibco), 1× Trace Elements A, B and C (Cellgro; #99-182-C1, #99-176-C1, #99-175-C1), 50 ug/ml Ascorbic Acid (Sigma, #A4034), 10 μg/ml Transferrin (Gibco, ##11107-018), 0.1 nM beta-mercaptoethanol, 8 ng/ml Fgf2 (Sigma, #F0291), 100 ng/ml Activin A (R&D Systems, #338-AC), Wnt3a (25 ng/ml, R&D Systems).
(b) A second defined differentiation media composed of; DMEM/F12, Fgf2 (10 ng/ml; Sigma, #F0291), Wnt3a (25 ng/ml, R&D Systems), Activin A (100 ng/ml, R&D Systems), bovine serum albumin (2%, Seriologicals, #82-047-3), transferrin (10 μg/ml, Gibco, ##11107-018), trace elements, P-mercaptoethanol could also be used as a base media to which specification signals could then be added.
Optimally, Wnt3a is included for the first 24-36 hours after switching to differentiation medium.
Assays for differentiation: as per previous filings and McLean et al., 2007 (Stem Cells).

### Description of the Results:

In order to demonstrate generation of authentic definitive endoderm from hESCs, it is important to demonstrate that extraembryonic lineages are not being produced since several of the markers used to identify DE (such as Sox17, GATA4,6, FoxA2) are also expressed by other cell types such as extraembryonic lineages. A feature of DE differentiation is that it forms following the transition through an intermediate cell type known as mesendoderm. This cell type typically expresses T, MixL1 and Wnt3a and it is important to show that presumptive DE forms by passing through a T+ state as described in McLean et al 2007 and D'Amour et al 2005.

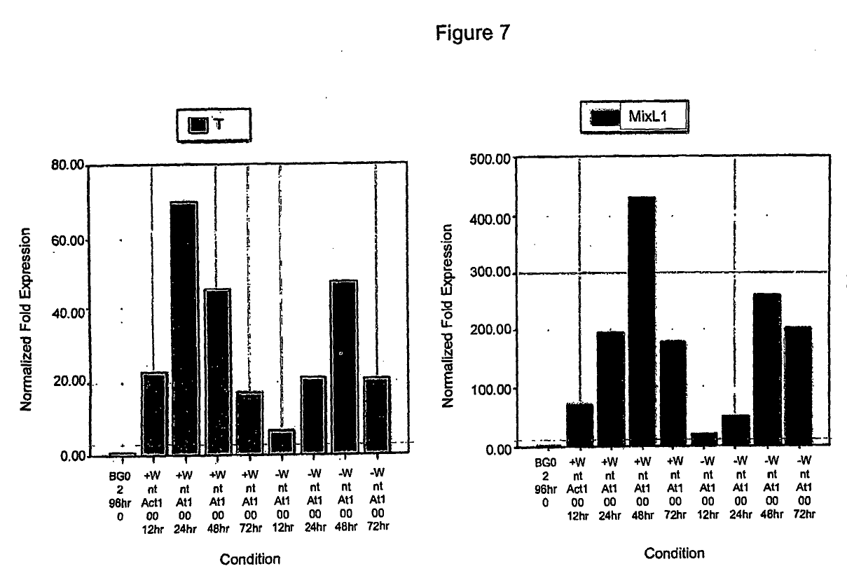

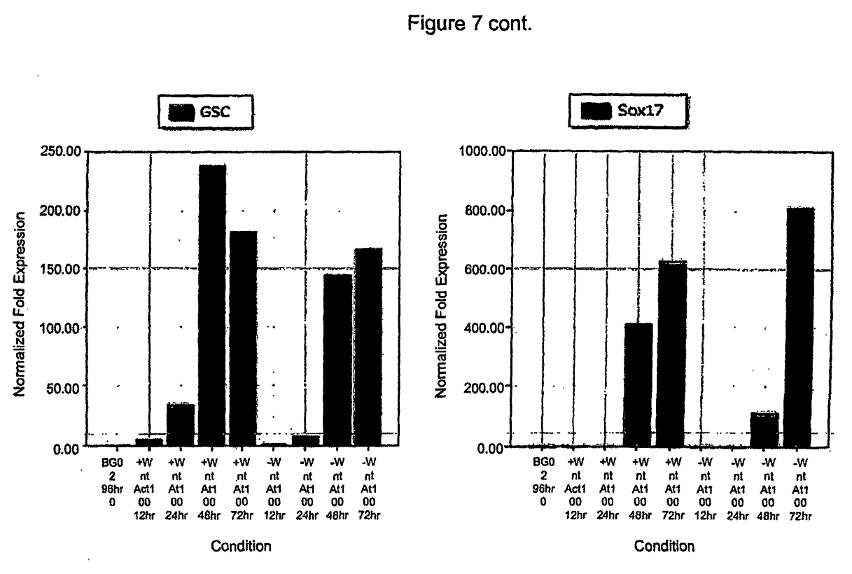

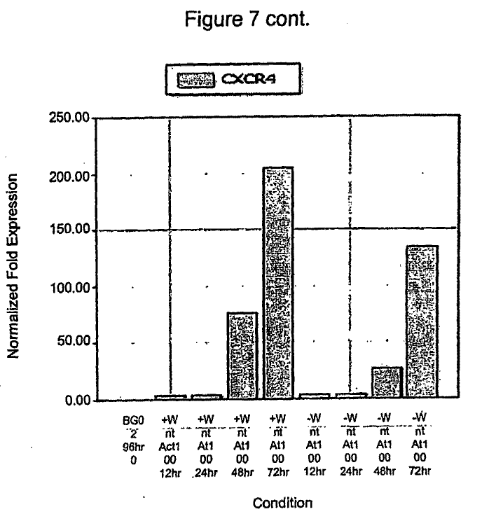

In this application we show that hESCs can be efficiently differentiated into CXCR4+Sox17+DE under feeder free conditions in defined media FIG. 7 shows the increase in T mRNA levels at 12 hours, showing maximum levels by 24 hours (˜70-fold induction over untreated hESCs). MixL1 transcripts peak at 48 hours ((>400-fold induction). Transcripts associated with DE such as Sox17, Gsc and CXCR4 increase up to 72 hours of treatment showing up to 800, 230 and 200 fold increases, respectively. Parallel experiments performed in the presence or absence of Wnt3a indicate that +Wnt3a conditions improves the amount of CXCR4 mRNA produced over 72 hours of treatment, but is not essential.

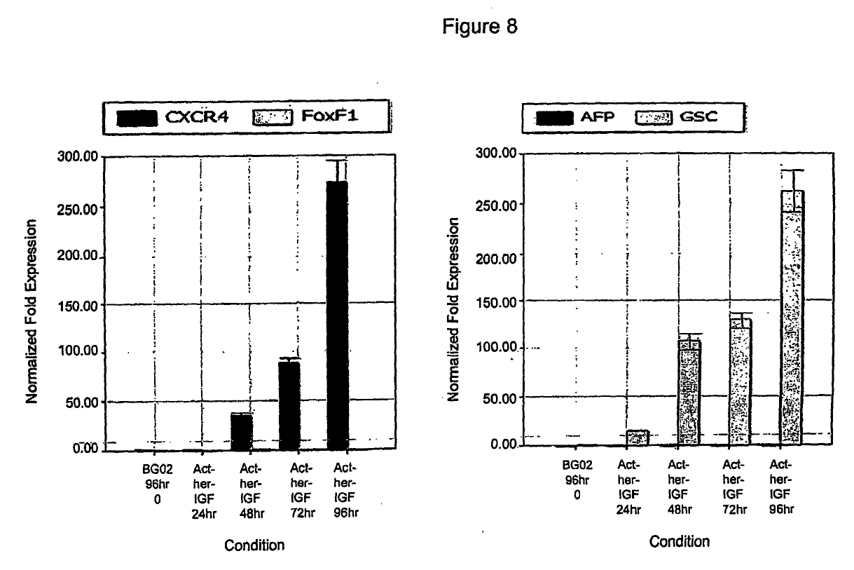

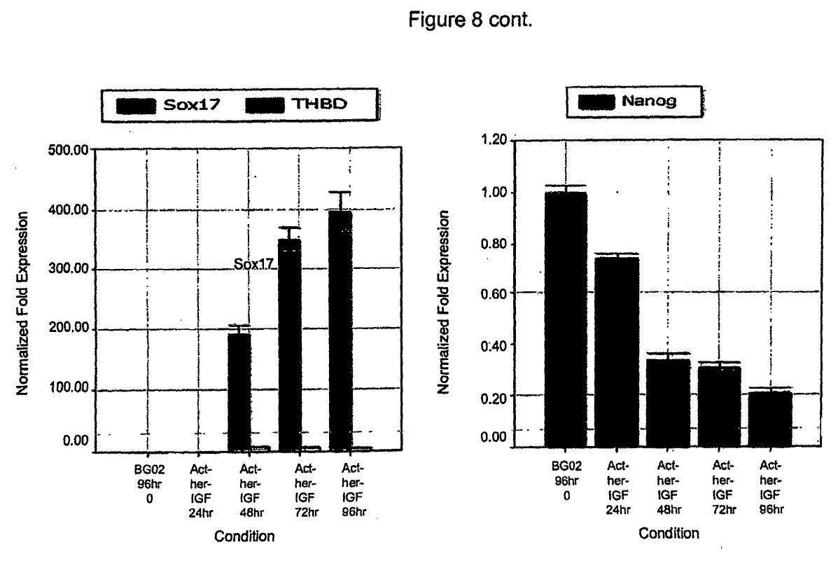

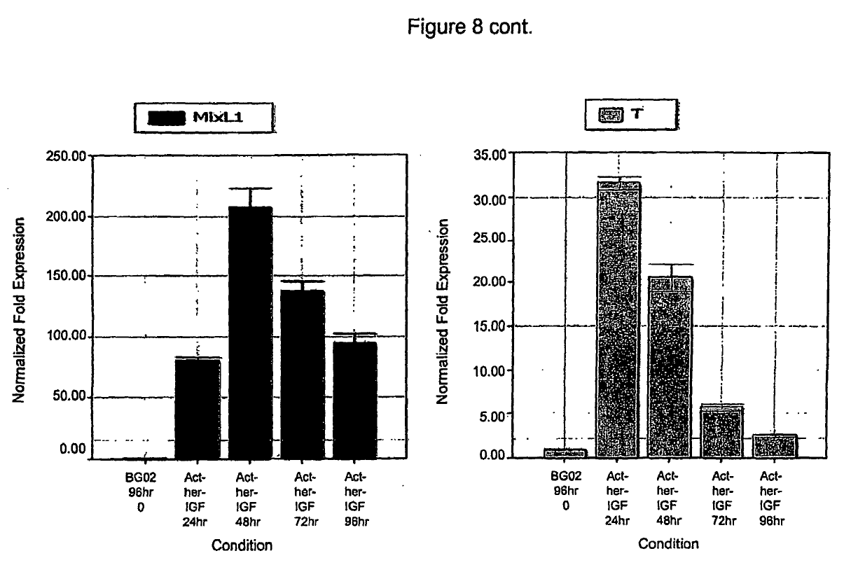

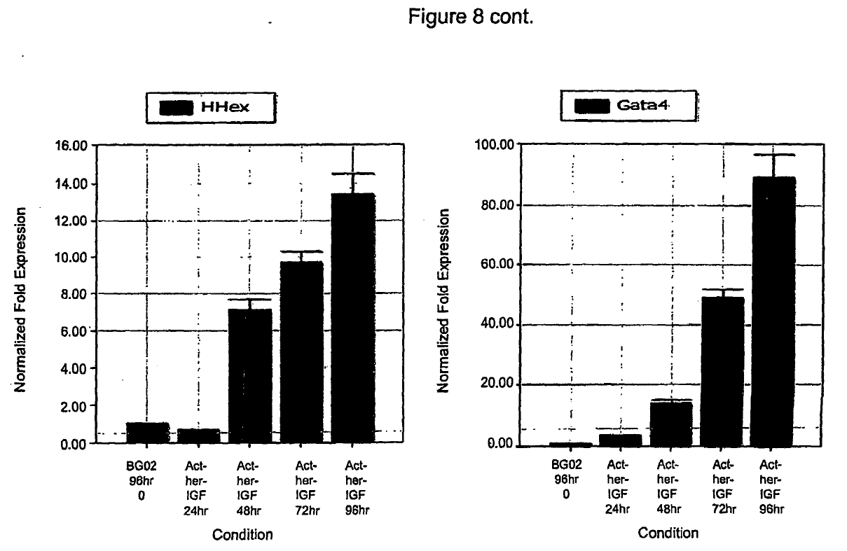

FIG. 8 shows Q-PCR analysis of marker transcripts to evaluate if extraembryonic (AFP, THBD) or mesoderm (FoxF1) was being produced. The analysis shows no increase in extraembryonic endoderm markers (AFP, THBD) over 96 hours of treatment, indicating that Sox17 expression is associated with DE formation. As DE transcripts increase such as Sox17 (˜400-fold), CXCR4 (˜275-fold), Gsc (˜260-fold), GATA4 (˜90-fold) levels of Nanog (an ESC marker) decrease. Increases in the levels of mesendoderm transcripts (T, MixL1) precede increases in DE transcripts by 24-48 hours, consistent with the cell population transitioning through mesendoderm on their way to becoming DE.

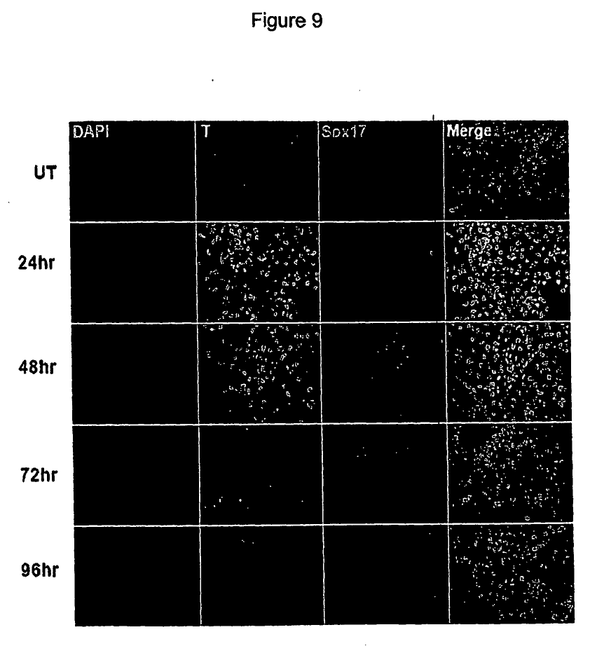

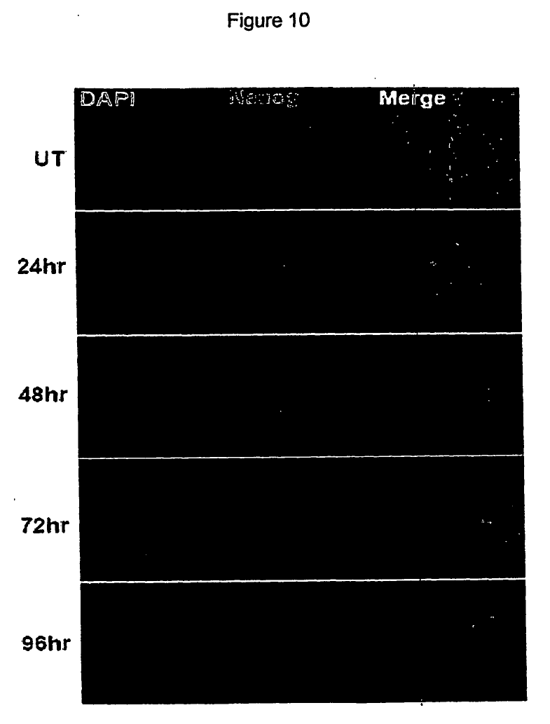

Analysis of the % cells differentiating into DE and mesendoderm was evaluated by immunocytochemistry of cells by probing with Sox17 and T antibodies, respectively (FIG. 9). At 24 and 48 hours after the switch to differentiation medium, almost 100% of cells stained positive with the T antibody, indicating that a near homogeneous population of mesendoderm was present at these time points. By 48 hours of treatment, the % of Sox17 positive cells began to increase and a number of Sox17-T double positive cells (˜20%) detected. By 72 hours the cultures consisted of >95% Sox17+ cells but with <5% of these being T+. After 96 hours, >95% of cells in these cultures stained positive for Sox17 with no detectable staining for T. Nanog staining remained high throughout cultures for the first 24 hours of differentiation but collapsed markedly from 24 hours through to 96 hours (FIG. 10).

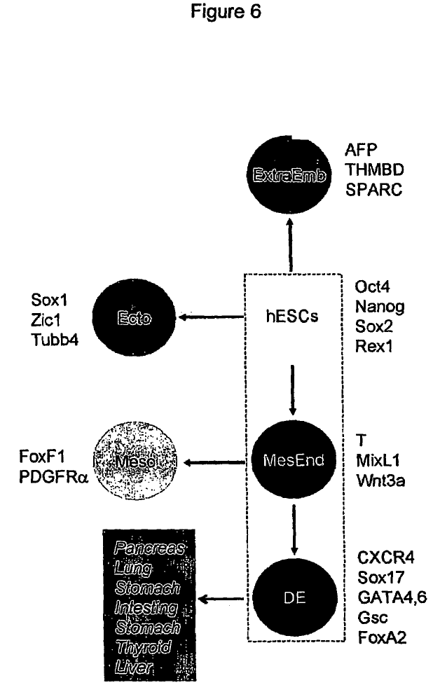

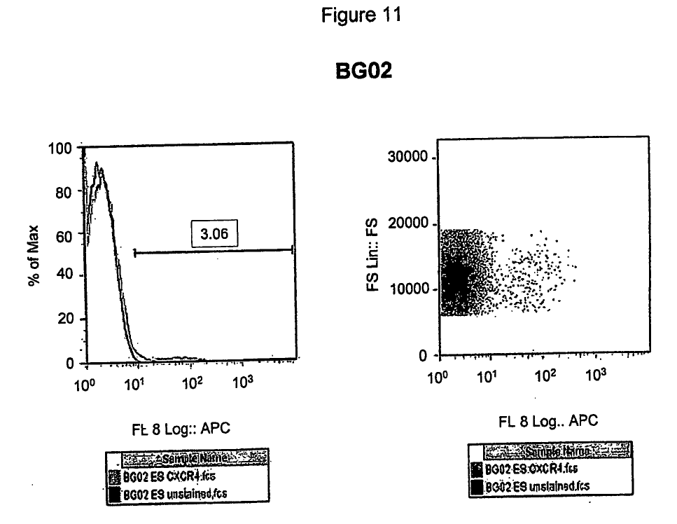

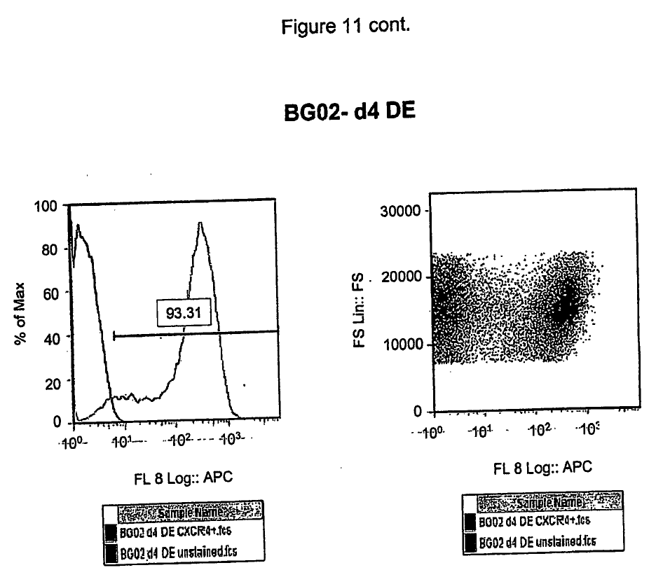

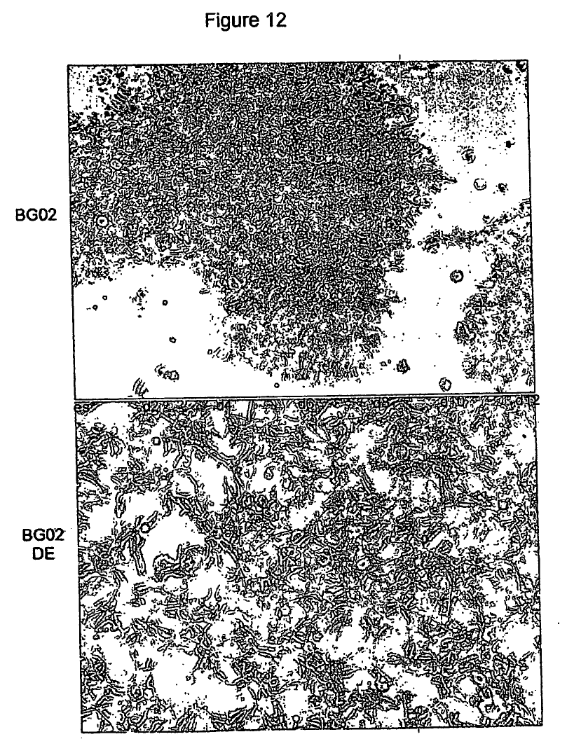

To confirm the high percentage of DE in these cultures we performed flow cytometry analysis of CXCR4 stained cells (FIG. 6). We consistently obtained cultures containing >93% CXCR4 positive cells by the methods described in the filing. This is superior to any other method reported to date. Bright field images of hESCs and DE generated under our conditions are shown (FIG. 11).

We have developed an improved method for the generation of DE from hESCs. This method has several advantages over previous methods including a more robust, reproducible culture system that is more appropriate for the development of cell therapeutics.

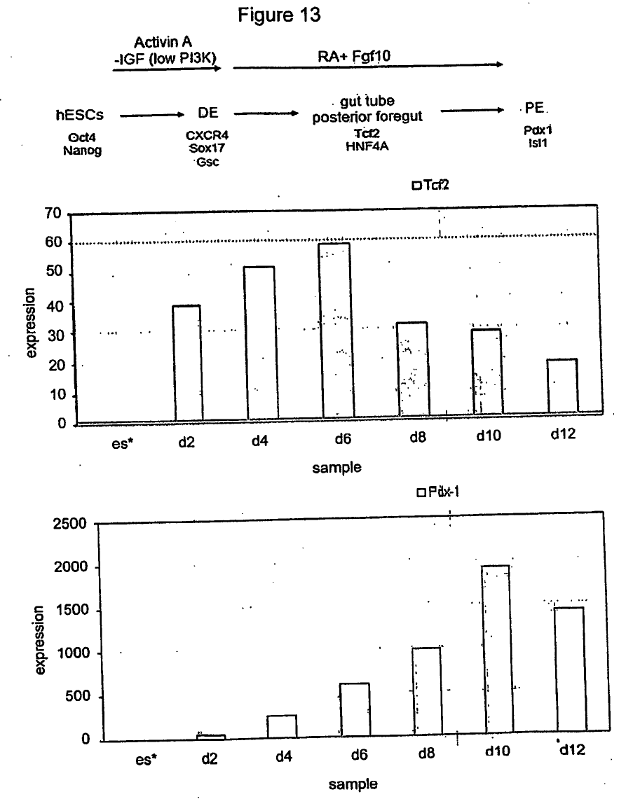

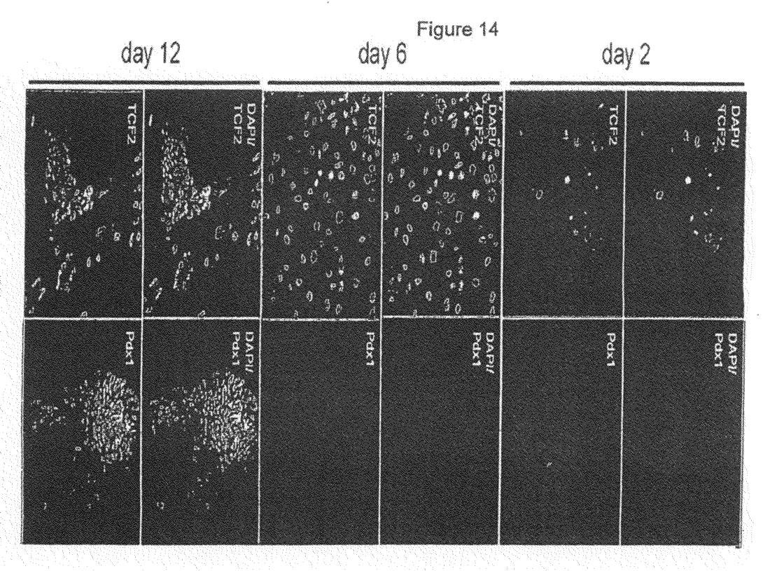

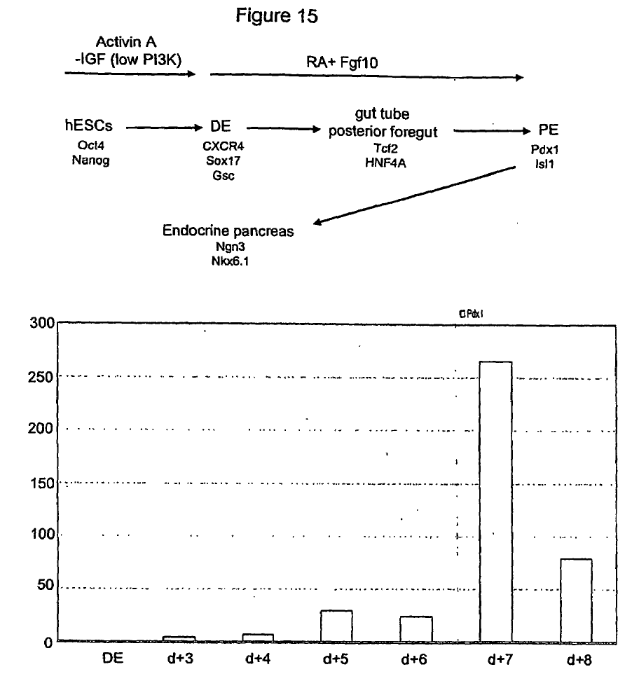

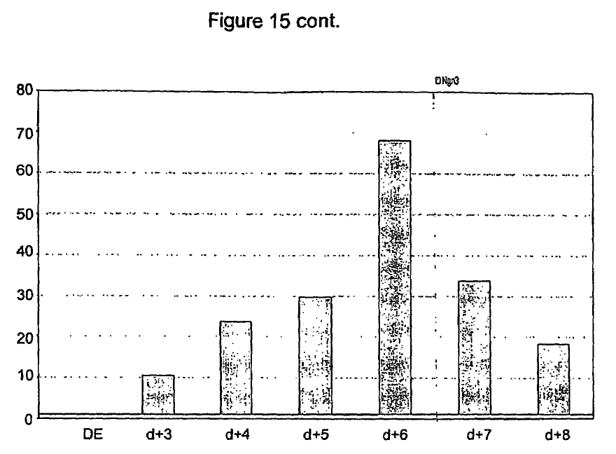

FIG. 8 shows the general strategy used to differentiate hESCs into DE and then posterior foregut/pancreatic endoderm (Pdx1+ cells). Increases in mRNA for the gut tube marker Tcf2 and the pancreatic endoderm/posterior foregut marker Pdx1 are shown over a 12 day time course after DE was replated in medium containing RA and Fgf10 (FIG. 13). Transcripts were as assayed by QRT-PCR. Tcf2 transcripts peak at ˜day 6, increasing ˜60-fold over levels in hESCs and Pdx1 transcripts peak at ˜day 10, showing an increase of almost 2,000 fold over levels in hESCs. ICC was then performed on DE plated in the presence of RA and Fgf10 for 2, 6 and 12 days (FIG. 14). Tcf2 positive cells were detected at day 2 of treatment (˜25% positive) but this increased to 100% by day 6 and decreased slightly by day 12 to 50%. Pdx1 positive cells were not detected at days 2 and 6 but the culture was >80% on day 12.

These general methods work for all cell lines tested including BG01, BG02, H7, H9.
Methods for Generation of Pancreatic Endoderm from Definitive Endoderm

Once CXCR4+DE is produced by one of the approaches described above, cells are passaged using accutase or collagenase (or similar) and plated on Matrigel (1:200; or similar matrix). DE is plated typically at 0.5×10⁶/60 mm dish in PE differentiation media (see below) for up to 12 days.

### Pancreatic Endoderm Differentiation Media:

(a) DMEM:F12 (Gibco), 2% BSA (Seriologicals, #82-047-3), 1× Pen/Strep (Gibco), 1× non-essential amino acids (Gibco), 1× Trace Elements A, B and C (Cellgro; #99-182-C1, #99-176-C1, #99-175-C1), 50 ug/ml Ascorbic Acid (Sigma, #A4034), 10 μg/ml Transferrin (Gibco, ##11107-018), 0.1 nM beta-mercaptoethanol, 10 ng/ml Activin A (R&D Systems. #338-AC), 10 ng/ml Heregulin beta (Peprotech; #100-03), 200 ng/ml LR-IGF (JRH Biosciences, #85580), all-trans retinoic acid (0.2-2.0 μM; Sigma-Aldrich), 50 ng/ml recombinant human Fgf10 (R&D Systems).
Use of other defined media formulations (see human ESC defined media above) is applicable to this differentiation step when supplemented with effective amounts of retinoic acid and Fgf10.

### Examples

To promote (efficient/controlled/specified outcomes) differentiation of hESCs, including the production of specific lineages including endoderm lineages, including but not restricted to definitive endoderm or pancreatic endoderm.
For therapeutic and non-therapeutic purposes
Same as above but for other (ie adult) stem cell or progenitor applications.
In cancer applications where tumor cells dedifferentiate, where cancer cells are stern-like cells and where differentiation of cancer cells doesn't occur.
In cases where progenitor or partially committed cells fail to differentiate in various disease states.
This invention can be used by itself through addition directly to HESC media, or in the presence of low serum or in conjunction with other factors including growth factors, such as cytokines, among others including TGF and superfamily members, among others.
As part of a drug screening where specific cell types need to be generated.

1. A method of producing human pancreatic endoderm cells from definitive endoderm cells comprising:
a. exposing human definitive endoderm cells to an effective amount of retinoic acid in a cell differential medium for a period of at least one day; and
b. stabilizing the differentiated cells obtained from step a by exposing said cells to a stabilizing medium in the absence of retinoic acid.

2. The method according to claim 1 wherein said definitive endoderm cells are obtained from human embryonic stem cells exposed to a P13K inhibitor.

3. The method according to claim 1 wherein said cell differential medium comprises retinoic acid at a concentration ranging from about 0.05 μg/ml to about 25 μg/ml.

4. The method according to claim 1 wherein said cell differential medium comprises retinoic acid at a concentration ranging from about 0.1 μg/ml to about 2 μg/ml.

5. The method according to claim 1 wherein said cell differentiation medium further includes fibroblast growth factor.

6. The method according to claim 1 wherein said cell differentiation medium further includes fibroblast growth factor 10 at a concentration ranging from about 2 μg/ml to about 100 μg/ml.

7. The method according to claim 1 wherein said cell differentiation medium further includes fibroblast growth factor 10 at a concentration ranging from about 2 μg/ml to about 100 μg/ml.

8. The method according to claim 1 wherein said cell differentiation medium is a basal cell medium comprising about 2% to about 20% fetal calf serum.

9. The method according to claim 1 wherein said cell differentiation medium is a basal cell medium comprising about 10% fetal calf serum.

10. The method according to claim 1 wherein said stabilizing medium is a basal cell medium comprising about 2% to about 20% fetal calf serum.

11. The method according to claim 1 wherein said stabilizing medium is a basal cell medium comprising about 10% fetal calf serum.

12. The method according to claim 1 wherein said exposing step occurs over a period of at least about 2 days.

13. The method according to claim 1 wherein said exposing step occurs over a period of at least about 4 days.

14. The method according to claim 1 wherein said exposing step occurs over a period of about 4 days.

15. The method according to claim 1 wherein said stabilizing step occurs over a period of at least about 1 day.

16. The method according to claim 1 wherein said stabilizing step occurs over a period of at least about 2 days.

17. The method according to claim 1 wherein said basal cell medium is a mixture of DMEM and F12.

18. The method according to claim 16 wherein said basal cell medium is a 50:50 mixtures of DMEM and F12.

19. The method according to claim 1 wherein said exposing step or said stabilizing step occurs wherein definitive endoderm cells or said differentiated cells are grown on a support comprising a differentiation protein.

20. The method according to claim 1 wherein said exposing step or said stabilizing step occurs wherein definitive endoderm cells or said differentiated cells are grown on a support comprising matrigel.

21. The method according to claim 1 wherein said exposing step and said stabilizing step occurs wherein said definitive endoderm cells or said differentiated cells are grown on a support comprising a differentiation protein.

22. A method of producing liver endoderm cells from definitive endoderm cells comprising:
a. exposing definitive endoderm cells to an effective amount of fibroblast growth factor in a cell differential medium for a period of at least one day; and
b. stabilizing the differentiated cells obtained from step a by exposing said cells to a stabilizing medium.

23. The method according to claim 21 wherein said fibroblast growth factor is fibroblast growth factor 10.

24. A method of producing pancreatic endoderm cells comprising:
a. producing definitive endoderm cells from human embryonic stem cells by exposing human embryonic stem cells to a basal cell medium comprising an effective amount of a P13K inhibitor as a differentiation agent;
b. stabilizing said definitive endoderm cells from step a;
c. exposing definitive endoderm cells after step b to an effective amount of retinoic acid in a cell differential medium for a period of at least one day; and
d. stabilizing the differentiated cells obtained from step a by exposing said cells to a stabilizing medium in the absence of retinoic acid.

25. A method of generating definitive endoderm cells from embryonic stem cells, under feeder cell-free conditions, comprising exposing plated embryonic stem cells to a defined media or MEF conditioned media using a growth matrix, and thereafter, exposing the stem cells to a differentiation media which is a defined media comprising effective amounts of Activin A, nodal, TGFβ or other TGF component and optionally, an inhibitor of PI3kinase signaling.

26. The method according to claim 24 wherein defined media includes an inhibitor of PI3K signaling.

27. The method according to claim 24 wherein said growth matrix is matrigel.

28. A method of generating definitive endoderm cells from embryonic stem cells, comprising exposing said exposed stem cells in the absence of feed cells to a differentiation media comprising elevated levels of Activin A, nodal or TNFβ and optionally, an inhibitor of PI3kinase signaling, wherein said differentiation media is a defined media free from fetal calf serum or KSR-type serum components.

29. The method of claim 24 wherein said definitive endoderm cells are produced at a level of at least about 90% from said embryonic stem cells.

30. The method according to claim 24 wherein said definitive endoderm cells are further differentiated into pancreatic endoderm cells.

31. The method according to claim 24 wherein said cells are human cells.

32. A method of generating pancreatic endoderm cells from human embryonic stem cells said method comprising generating definitive endoderm cells from embryonic stem cells, under feeder cell-free conditions, comprising exposing embryonic stem cells to a defined media or MEF conditioned media using a growth matrix, and thereafter, exposing the stem cells to a differentiation media which is a defined media comprising effective amounts of a SMAD pathway activator and optionally, an inhibitor of PI3kinase signaling for a period of about 3-6 days to produce definitive endoderm cells and thereafter exposing said definitive endoderm cells to a defined media comprising an effective amount of retinoic acid and optionally, an effective amount of FGF10 for a further period of about 5-12 days, preferably 8-10 days to produce pancreatic endoderm cells.

33. The method according to claim 32 wherein said SMAD pathway activator is selected from the group consisting of Activin A, nodal, TGFβ, TGF component or mixtures thereof.

34. The method according to claim 32 wherein said media further includes an effective amount of wnt3a.

35. The method according to claim 34 wherein said growth matrix is madrigel.
